(19) United States
(12) Patent Application Publication
(10) Pub. No.: US 2022/0089574 A1
(43) Pub. Date: Mar. 24, 2022

(54) CXCR4 gamete molecular structure and method thereof

(71) Applicants: MING-HSIN LI, Taoyuan City (TW); SHENG-NAN LO, Taoyuan (TW); SHIH-WEI LO, Taoyuan (TW); SHIH-YING LEE, Taoyuan (TW); SU-JUNG CHEN, Taoyuan (TW); WEI-LIN LO, Taoyuan (TW); MING-WEI CHEN, Taoyuan (TW); YUAN-RUEI HUANG, Taoyuan (TW)

(72) Inventors: MING-HSIN LI, Taoyuan City (TW); SHENG-NAN LO, Taoyuan (TW); SHIH-WEI LO, Taoyuan (TW); SHIH-YING LEE, Taoyuan (TW); SU-JUNG CHEN, Taoyuan (TW); WEI-LIN LO, Taoyuan (TW); MING-WEI CHEN, Taoyuan (TW); YUAN-RUEI HUANG, Taoyuan (TW)

(21) Appl. No.: 17/027,748

(22) Filed: Sep. 22, 2020

(51) Int. Cl. C07D 403/12; C07F 5/00

(52) U.S. Cl. CPC C07D 403/12; A61K 51/0482; C07F 5/003

(57) ABSTRACT

The structure and preparation method of a compound D-L2(-B)-L1-A is disclosed, A is a physiologically and pharmacologically active molecule that physically binds to a specific biological molecule or receptor; L1 is a variable structure with the molecular connection activity at both ends connecting to A and L2; L2 is a variable structure, which has three-terminal molecular connection activity connecting to L1, D and B; B is a physiologically and pharmacologically active molecule that binds to albumin, changing the A molecule in the body cyclic characteristics; D is a polycarboxylic macrocyclic structure that binds to radioisotopes; D-L2(-B)-L1-A compound can be used to express chemokine receptor 4 (CXCR4) receptors on cells, tissues, and/or organs, and after binding with radionuclides, it is suitable for detection of CXCR4 protein binding in vitro, detection of CXCR4 cell binding in vitro or detection of CXCR4 expression in in vivo animal imaging.

### BACKGROUND OF THE INVENTION

### 1. Field of the Invention

The present invention relates to a compound for improving insufficient accumulation of existing drugs in tumors, especially to the compound with a molecular structure of D-L2(-B)-L1-A or a pharmaceutically acceptable salt thereof. The present invention further relates to a method for the making and using of said compound.

### 2. Description of Related Art

It is well known that, among many factors, CXCR4 and chemokine ligand 12 (CXCL12) represent an important axis of the tumor microenvironment that influences tumor growth. Cell proliferation, angiogenesis, tumor invasion and migration are important aspects that interact with the fibroblasts, endothelial cells and immune cells in the tumor microenvironment to protect and support niche cancer cells. These interactions have an important impact on tumor growth. CXCR4 is a G protein-coupled receptor that penetrates the membrane seven times, and is abundantly expressed on the surface of tumor cells. When the tumor microenvironment is in a relatively hypoxic state, tumor cells' CXCR4 will be expressed in large quantities and CXCL12 will increase in the tumor microenvironment. The combination of CXCR4 and CXCL12 promotes tumor cell proliferation, and CXCL12 induces endothelial cells to promote angiogenesis; in addition, CXCL12 is expressed in large quantities in vital organs such as bone marrow, lungs, and livers.

Therefore, when CXCR4 are expressed in tumor cells, these receptors will be targeted to said vital organs. Furthermore, CXCR4-CXCL12 has shown to critically influence the development of many types of tumors, including lymphoma, gastrointestinal and pancreatic tumors, ovarian cancer, breast cancer, prostate cancer, lung cancer, multiple myeloma, renal cell carcinoma, melanoma, brain cancer and soft tissue sarcoma.

The cancers associated with the above-mentioned CXCR4 in the American Cancer Institute's prediction of common cancers in 2020 revealed that the incidence in female breast cancer was 15.3%, and 6% of newly diagnosed stages were remote metastases, and the 5-year relative survival rate of patients in this state was only 28.1%. The incidence in lung and bronchial cancer was 12.7%, and more than half of newly diagnosed stages were non-original, and there were 57% remote metastasis and 22% lymphatic metastasis, and 4% were unknown, and the 5-year relative survival rates of patients with their respective states were only 31.7%, 5.8% and 8.3%, respectively.

The incidence in male prostate cancer was 10.60%, and 6% of newly diagnosed stages were remote metastases, and the 5-year relative survival rate of patients in this state was only 30.2%. The incidence in colorectal cancer was 8.2%, and 22% of newly diagnosed stages were remote metastases and 4% were unknown, and the 5-year relative survival rates of patients in this state were only 14.3% and 37.5%, respectively. The incidence in melanoma skin cancer was 5.6%, and 4% of newly diagnosed stages were remote metastases, and the 5-year relative survival rate of patients in this state was only 27.3%.

The incidence in kidney cancer was 4.1%, and the newly diagnosed stages had 16% of remote metastases and 3% were unknown, and the 5-year relative survival rates of patients in this state were only 13% and 42.3%, respectively; the incidence in pancreatic cancer was 3.2%, newly diagnosed stage had 11% in situ, 30% were lymphatic metastases, 52% were remote metastases and 7% were unknown, and the 5-year relative survival rates of patients were only 39.4%, 13.3%, 2.9% and 6.1%, respectively.

The incidence in liver and intrahepatic cholangiocarcinoma was 2.4%, the newly diagnosed stage had 44% in situ, 27% were lymphatic metastasis, 18% were remote metastasis, and 11% were unknown, and the 5-year relative survival rates of patients were only 34.2%. 12%, 2.5%, and 7%, respectively. The incidence in gastric cancer was 1.5%, the newly diagnosed stages included 26% lymphatic metastases, 36% remote metastases, and 10% were unknown, and the 5-year relative survival rates of patients were only 32%, 5.5% and 23.4%, respectively. The incidence in brain and other nervous system cancers was 1.3%, and the newly diagnosed stage was 77% in situ, 15% were lymphatic metastases, 2% were distal metastases, and 6% were unknown, and the 5-year relative survival rates of patients were only 35.3% and 20.3%, 32.7% and 29%, respectively.

The incidence in female ovarian cancer was 1.2%, and the newly diagnosed stage had 58% remote metastases and 5% were unknown, and the 5-year relative survival rates of patients were only 30.2% and 25.5%, respectively. The incidence rate of esophageal cancer was 1%, and the newly diagnosed stage 18% in situ, 33% were lymphatic metastases, 39% were remote metastases, and 10% were unknown. The 5-year relative survival rates of patients were only 47.1%, 25.2%, 4.9% and 12.8%, respectively. The incidence in soft tissue cancer was 0.7%, and 15% of newly diagnosed stages were remote metastases, and the 5-year relative survival rate of patients was only 15.4%.

The incidence in small intestine cancer was 0.6%, and 27% of newly diagnosed stages were remote metastasis, and the 5-year survival rate of patients was only 42.2%. The American Cancer Institute's statistics show that many newly diagnosed stages have undergone remote metastases and the 5-year relative survival rate of patients is less than 50%, which revealed the poor survival and prognosis of patients after diagnosis. It highlights the need for more advantageous imaging or visualization tools to accurately identify the cancer status before the occurrence of remote metastases.

The current CXCR4 peptide inhibitors used in the performance of CXCR4 tumor imaging technology are mainly divided into two categories: TN14003 and FC 131 and their modified derivatives, as described below.1. TN14003 and its derivatives: TN14003 is a peptide composed of 14 amino acids, of which the 4th and 13th Cys amino acids form disulfide bonds via side chains, making them a cyclic peptide, this category includes: (1) Ac-TZ14011: Ac-TZ14011 uses the 8th amino acid lysine and DTPA to join the In-111 marker, In-111-DTPA-Ac-TZ14011, which is used in the animal model of CXCR4 pancreatic cancer metastasis. Extract the animal tissues and organs at a specific time point, and count the radioactivity. The tumor is 0.51±0.08, 0.20±0.03 and 0.14±0.03 at 1, 6, and 12 hours, respectively, in unit: % injection dose/organ tissue weight, % ID/g.

(2) T140: TN14003 uses the first arginine and N-succinimidyl 4-18F-fluorobenzoate reaction marker F-18 (4-F-18-T140) for the animal model of CXCR4 tumors, and the animals tissue and organ are harvested after 3 hours of distribution, and read the radioactivity, the distribution of the tumor is 2.3±0.7% ID/g. (3) Ac-TZ14011-MSAP: TN14003 amino acid is replaced with Ac-TZ14011, modified by MSAP, and labeled with In-111, In-111-DTPA-Ac-TZ14011-MSAP, which is used in breast cancer animal model expressing CXCR4 mode, the tumor distribution within 24 hours after administration was 1.10±0.60% ID/g; and dimer (Ac-TZ14011)₂ was modified by MSAP and labeled with In-111, In-111-DTPA-(Ac-TZ14011)₂-MSAP, which is used in breast cancer animal model expressing CXCR4, the distribution of the tumor within 24 hours after administration is 0.57±0.19% ID/g; and tetramer (Ac-TZ14011)₄ is modified by MSAP and labeled with In-111. In-111-DTPA-(Ac-TZ14011)₄-MSAP, which is used in breast cancer animal model expressing CXCR4, the distribution of the tumor within 24 hours after administration is 0.42±0.10% ID/g.

(4) DOTA-NFB/NOTA-NFB: TN14003 connects the first arginine with DOTA or NOTA, and then uses the Cu-64 for labeling to obtain Cu-64-DOTA-NFB or Cu-64-NOTA-NFB, which is used in the CXCR4 tumor expressing animal model, and the tumor accumulation for 1 and 4 hours captured by microPET image is 4.09±1.37 and 3.58±0.67% ID/g (Cu-64-DOTA-NFB); 4.34±1.00 and 4.38±0.68% ID/g (Cu-64-NOTA-NFB), and animal tissues and organs were harvest at a specific time point, and measure the radioactivity. The tumor is 4.98±0.89 (Cu-64-DOTA-NFB) and 4.55±0.66 (Cu-64-NOTA-NFB) % ID/g.(5) T140-2D: TN14003 is connected to the 7th and 8th amino acid lysine respectively with DOTA and Cu-64 labeled Cu-64-T140-2D, which is used in the animal model of CXCR4 tumor. The animal tissues and organs were harvested in 4 hours, and the radioactivity was calculated and the tumor distribution was 3.18±0.51% ID/g.

(6) CCIC16 (NO2A-TN14003): TN14003 combines the first arginine with one of the three carboxylic acids on the NOTA structure, and then uses the Ga-68 to label to obtain Ga-68-CCIC16 for use in the CXCR4 tumor expressing animal model. The tumor accumulation was 3.73±1.12% ID/mL by dynamic PET image capture within 1 hour of distribution, and animal tissues and organs were harvested within 1 hour of distribution, and the radioactivity was calculated and the tumor distribution was 5.33±1.84% ID/g.

2. FC 131 and its derivatives: FC 131 is a peptide composed of 5 amino acids, which is made into a cyclic structure with amide bonds, this category includes: (1) Pentixafor: replace the second amino acid Arg of FC 131 with NMe-Orn, and join the ring structure and DOTA with 4-aminomethyl benzoic acid, and then use the Ga-68 to label to obtain Ga-68-pentixafor for use in the CXCR4 tumor expressing animal model. The animal tissues and organs are harvested at a specific time point and the radioactivity is calculated. The distribution of the tumor at 10 minutes, 1 hour and 2 hours is 4.55±1.10 (n=5), 6.16±1.16 (n=8) and 4.63±1.54 (n=3) % ID/g, respectively.

(2) CCIC-0007: replace the second amino acid Arg of FC 131 with Orn, and connect the structure of PEG2-O—NH2, and then use [F-18]FBA to label to obtain F-18-CCIC-007 for use in the CXCR4 expressing tumor cells, the results of the binding of competitive cells showed that the IC₅₀ was 0.80 μM, and there is no animal tumor model data. (3) NOTA-pentixather: replace the first amino acid Tyr of FC 131 with 3-iodo-Tyr and the second amino acid Arg with NMe-Orn, and use of 4-aminomethyl benzoic acid to join the ring structure and NOTA, and then use the F-18-Al to label to obtain F-18-Al-NOTA-pentixather for use in the CXCR4 expressing animal tumor model. The animal tissues and organs were harvested at a specific time point and the radioactivity is counted. The tumor distribution of 1 hour is 14.0±1.0 (n=4) % ID/g.

(4) HYNIC-peptide: replace the second amino acid Arg of FC 131 with Orn, connect to the HYNIC structure and label with Tc-99m to obtain Tc-99m-HYNIC-peptide, which is used for CXCR4 expressing animal tumor model, and the animal tissues and organs were harvested at a specific time point and count the radioactivity. The distribution of 15 minutes, 30 minutes, 1 hour, 2 hours and 24 hours at the tumor is 2.74±0.47 (n=3), 0.95±0.05 (n=3), 0.18±0.06 (n=3), 0.09±0.03 (n=3) and 0.03±0.01 (n=3) % ID/g, respectively.

(5) Pentixather: replace the first amino acid Tyr of FC 131 with 3-iodo-Tyr and the second amino acid Arg with NMe-Orn, join the ring structure and DOTA with 4-aminomethyl benzoic acid, and then use Lu-177-pentixather to react with CXCR4 cells for 1 hour, the results showed that the intracellular radioactivity was lower than 2% of the total dose, and lower than 6% for the cell membrane of the total dose. In addition, when applied to the animal model of lymphoma with CXCR4 express, the distribution of tumor at 1, 6, 48, 96 hours and 7 days after administration is 12.4±3.7, 6.79±0.68, 3.27±0.41, 2.07±0.12 and 2.06±0.37% ID/g, respectively. With In-111 labeling to obtain In-111-pentixather in the animal mode, the distribution of tumors at 1, 4, 24, 48, 72 and 96 hours after administration can be seen as 3.24, 1.54, 0.48, 0.5, 0.24 and 0.25% ID/g, respectively.

The above-mentioned CXCR4 peptide inhibitors are used for the imaging of CXCR4-expressing tumors. Among them, the faster development process such as Ga-68-pentixafor has been used in tumor patients, such as: multiple myeloma, lymphoma, acute myelogenous leukemia, small cell lung cancer, non-small cell lung cancer, aggressive breast duct carcinoma and glioblastoma. In addition, Lu-177-pentixather is also used in patients with multiple myeloma. However, from pre-clinical studies, due to different nuclei or modification of inhibitors or different sampling time points for inhibitors labeled with radioactivity, the highest value of radioactivity accumulated in the tumor is 14.0±1.0% ID/g with 18-labeled NOTA-pentixather, the second highest value of 12.4±3.7% ID/g is the Lu-177 labeled pentixather, and the accumulation of other inhibitors in the tumor is relatively low. Therefore, there is an urgent need to break through the accumulation barrier of inhibitors in tumors. By increasing the accumulation of inhibitors in tumors, it would be possible to reduce the repetition of use.

### SUMMARY OF THE INVENTION

The structural difference between the CXCR4 inhibitor pentixather and pentixafor lies in the substitution of iodine molecules in tyrosine, and the combination of pentixather and Lu-177 in radionuclides. Therefore, pentixather is a potential radionuclide inhibitor that can kill cells. The effect of radioactive inhibitors on tumor accumulation has a great impact on the application, including: the radioactive concentration given by the radioactive inhibitor, and even the number of times the radioactive inhibitor is given. The molecular structure D-L2(-B)-L1-A compound of the present invention or a pharmaceutically acceptable salt thereof comprises CXCR4 binding active structure A, albumin binding active structure B and radioactive chelator structure D, using L1 and L2 to combine 3 different functional structures and to connect them to improve the shortcomings of insufficient accumulation of the existing drugs in tumors.

### BRIEF DESCRIPTION OF THE DRAWINGS

FIG. 1 shows the structure and synthesis method of compound I-01(-I-01)-I-17 of the present invention;

FIG. 2 shows the structure and synthesis method of the compound I-01(-I-01)-I-01 of the present invention;

FIG. 3 shows the structure and synthesis method of the compound I-01(-II-01)-II-01 of the present invention;

FIG. 4 shows the structure and synthesis method of the compound I-01(-II-01)-II-08 of the present invention;

FIG. 5 shows the structure and synthesis method of the compound II-03(-II-08)-I-17 of the present invention;

FIG. 6 shows the structure and synthesis method of the compound II-01(-II-01)-I-01 of the present invention;

FIG. 7 shows an analysis of labeling efficiency and radiochemical purity of the compounds of the present invention;

FIG. 8 shows a radiation thin layer analysis of the compounds of the present invention;

FIG. 9 shows a radiation high pressure liquid chromatography of the compound of the present invention

FIG. 10 shows the radioactive labeling of the compound D-L2(-B)-L1-A of the present invention applied to animals in vivo imaging.

### DETAILED DESCRIPTION OF THE PREFERRED EMBODIMENTS

In an embodiment, by labeling the pentixather, D-L2(-B)-L1-A derivative I-01(-I-01)-I-17 and I-01(-I-01)-I-01 with In-111 to confirm the accumulation of radioactive compound with the tumor animal imaging:(1) In-111 labeled pentixather was distributed and accumulated in the tumor for 1 hour to be 3.24% ID/g, the radioactive concentration per unit weight of tissues/organs accounted for the percentage of the total injected radioactive concentration, and the In-111 labeled derivative revealed the accumulation of I-01(-I-01)-I-17 and I-01(-I-01)-I-01 in the tumor after 1 hour was 7.87% ID/g and 5.47% ID/g, respectively. The accumulation value of radiolabeled derivatives in tumors is higher than that of pentixather. It proves that the molecular structure of the present invention has increased the accumulation effect of CXCR4 inhibitors in tumors.

(2) In-111-labeled pentixather is distributed over time for 24 hours, and the accumulation value at the tumor has been reduced to 1% ID/g. In-111-labeled I-01(-I-01)-1-17 and I-01 (-I-01)-I-01 of the present invention was distributed over time for 24 hours, and the accumulation value at the tumor was still greater than 1% ID/g. Therefore, the structure of the present invention prolongs the accumulation time of CXCR4 inhibitors in tumors. The compound of the general formula D-L2(-B)-L1-A and a pharmaceutically acceptable salt thereof of the present invention, in which the structure A is cyclo, D-3-iodo-Tyr-[NMe]D-Orn(-L1-)-Arg-2-Nal-Gly, the amide bond is formed by the branched amino group —NH2 of Orn in the molecule and the carboxylic acid group —COOH of L1. The L1 structure having the function of connecting both ends, and there are two types I and II, of which L1 type I is represented by L1-I, and L1 type II is represented by L1-II. The L2 structure having a three-terminal connection function, and there are two types I and II, of which the L2 type I is represented by L2-I, and the L2 type II is represented by L2-II; the type I (L1-I) provides a carboxylic acid group (—COOH) at one end, and the other end provides an amino group (—NH2), in which the carboxylic acid group end generates an amide bond with the structure A, and the other end of the amino group generates an amide bond with any molecule of L2-I or L2-II.

L1-I is selected from one of the molecular structures of groups 1 to 26 shown in Table 1;

TABLE 1
L1-I structure

| | |
|---|---|
| 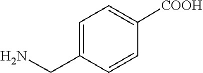 [SMILES: NCc1ccc(C(=O)O)cc1] | 1 |
| 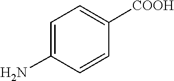 | 2 |
| 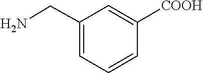 [SMILES: NCc1cccc(C(=O)O)c1] | 3 |
| 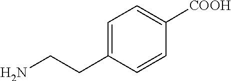 [SMILES: NCCc1ccc(C(=O)O)cc1] | 4 |
| 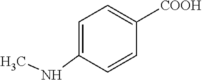 | 5 |
| 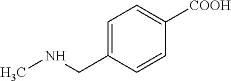 | 6 |
| 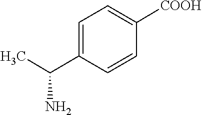 | 7 |
| 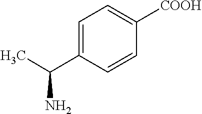 | 8 |
| 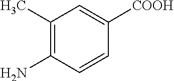 | 9 |
| 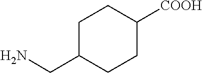 [SMILES: NCC1CCC(C(=O)O)CC1] | 10 |
| 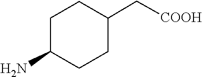 | 11 |
| 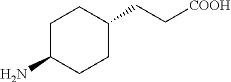 [SMILES: N[C@H]1CC[C@H](CCC(=O)O)CC1] | 12 |
| 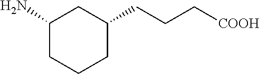 [SMILES: N[C@H]1CCC[C@@H](CCCC(=O)O)C1] | 13 |
| 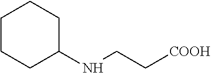 [SMILES: O=C(O)CCNC1CCCCC1] | 14 |
| 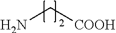 | 15 |
| 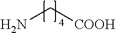 | 16 |
| 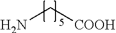 | 17 |
| 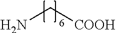 | 18 |
| 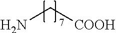 | 19 |
| 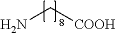 | 20 |
| 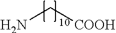 | 21 |
| 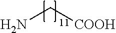 | 22 |
| 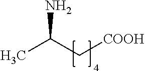 | 23 |
| 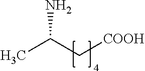 | 24 |
| 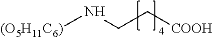 | 25 |
| 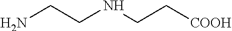 | 26 |
| — | — |

L1 Type II (L1-II) provides carboxylic acid groups (—COOH) at both ends, one of which is carboxylic acid group and structure A to form amide bond, and the other end of which is carboxylic acid group and any molecule of L2-I to form amide bond, and L1-II is selected from one of the molecular structures of groups 1 to 9 in Table II;

TABLE 2
L1-II structure

| | |
|---|---|
| 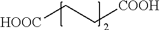 | 1 |
| 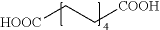 | 2 |
| 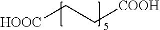 | 3 |
| 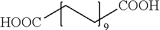 | 4 |
| 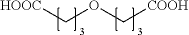 | 5 |
| 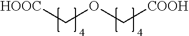 | 6 |
| 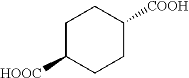 [SMILES: O=C(O)[C@H]1CC[C@H](C(=O)O)CC1] | 7 |
| 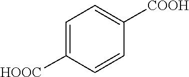 [SMILES: O=C(O)c1ccc(C(=O)O)cc1] | 8 |
| 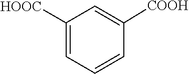 [SMILES: O=C(O)c1cccc(C(=O)O)c1] | 9 |

L2 has a three-terminal connection function with two types I and II. L2 type I (L2-I) provides α-carboxylate (—COOH), α-amino (—NH2) and branched amino (—NH2) molecule, in which the α-amino group and D produce an amide bond, the branched amino group and B (BI) produce an amide bond, and the α-carboxylic acid group end forms an amide bond with any structure of L1-I; also it is the α-amino group and D that produce an amide bond, the branched amino group and any structure of L1-II produce an amide bond, the α-carboxylic acid group end and B-II form an amide bond; also it is a branched amino group and D produce an amide bond, the α-amino group and any structure of L1-II produce an amide bond, the α-carboxylic acid group end forms an amide bond with B-II, where L2-I is selected from one molecular structures of the group 1 to 3 in Table 3;

TABLE 3
L2-I structure

| | |
|---|---|
| 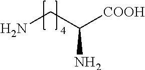 | 1 |
| 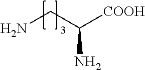 | 2 |
| 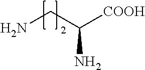 | 3 |

L2 Type II (L2-II) is to provide α-carboxylic acid groups (—COOH), branched carboxylic acid groups (—COOH) and α-amino (—NH2) molecules, in which α-amino terminal and D produce an amide bond, the α-carboxylic acid group end and B (B-II) form an amide bond, and the branched chain carboxylic acid group end forms an amide bond with any structure of L1-I, also it is the α-amino end and D to produce an amide bond, the branched carboxylic acid group end and B (B-II) to produce an amide bond, the α-carboxylic acid group end and any structure of L1-I form an amide bond, where L2-II is selected from one of the molecular structures of groups 1 to 4 in Table 4:

TABLE 4
L2-II structure

| | |
|---|---|
| 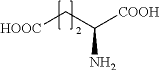 | 1 |
| 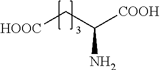 | 2 |
| 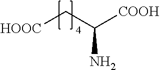 | 3 |
| 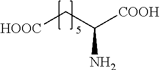 | 4 |

Where B is connected with L2, and B has two types I and II as shown in the table 5 and 6, where the BI structure has a carboxylic acid group (—COOH) to produce an amide bond with L2-I, and BI is selected from one of the molecular structure group 1 to 12 in Table 5;

TABLE 5

| Item | B-I structure |
|---|---|
| 1 | 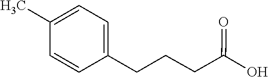 |
| 2 | 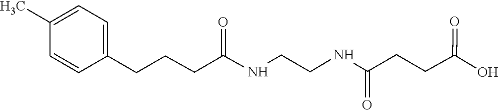 [SMILES: Cc1ccc(CCCC(=O)NCCNC(=O)CCC(=O)O)cc1] |
| 3 | 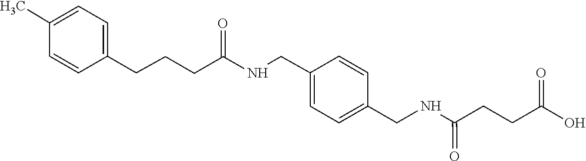 [SMILES: Cc1ccc(CCCC(=O)NCc2ccc(CNC(=O)CCC(=O)O)cc2)cc1] |
| 4 | 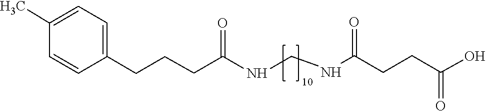 |
| 5 | 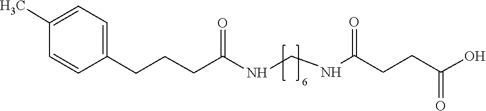 |
| 6 | 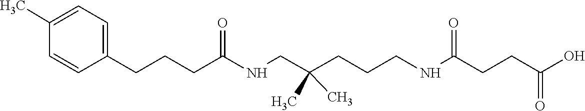 |
| 7 | 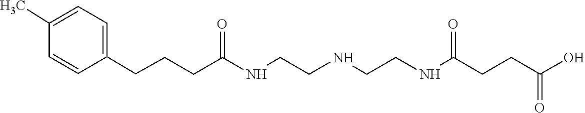 [SMILES: Cc1ccc(CCCC(=O)NCCNCCNC(=O)CCC(=O)O)cc1] |
| 8 | 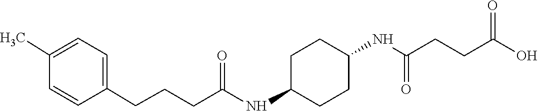 [SMILES: Cc1ccc(CCCC(=O)N[C@H]2CC[C@H](NC(=O)CCC(=O)O)CC2)cc1] |
| 9 | 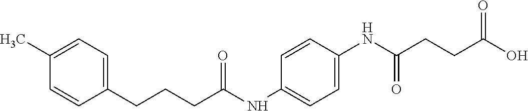 [SMILES: Cc1ccc(CCCC(=O)Nc2ccc(NC(=O)CCC(=O)O)cc2)cc1] |
| 10 | 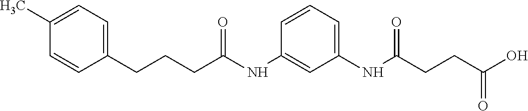 [SMILES: Cc1ccc(CCCC(=O)Nc2cccc(NC(=O)CCC(=O)O)c2)cc1] |
| 11 | 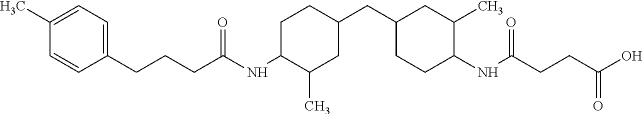 [SMILES: Cc1ccc(CCCC(=O)NC2CCC(CC3CCC(NC(=O)CCC(=O)O)C(C)C3)CC2C)cc1] |
| 12 | 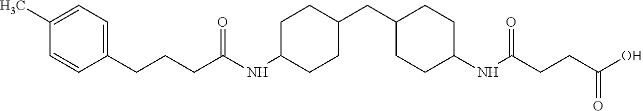 [SMILES: Cc1ccc(CCCC(=O)NC2CCC(CC3CCC(NC(=O)CCC(=O)O)CC3)CC2)cc1] |

B-II has an amino group (—NH2) to form an amide bond with L2-II, and B—II is selected from one of the molecular structures of groups 1 to 11 in Table 6;

TABLE 6

| Item | B-II structure |
|---|---|
| 1 | 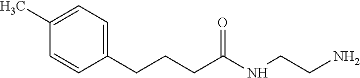 [SMILES: Cc1ccc(CCCC(=O)NCCN)cc1] |
| 2 | 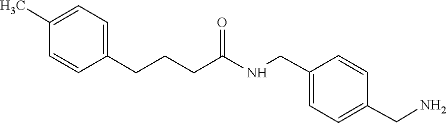 [SMILES: Cc1ccc(CCCC(=O)NCc2ccc(CN)cc2)cc1] |
| 3 | 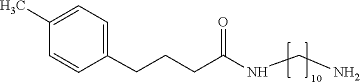 |
| 4 | 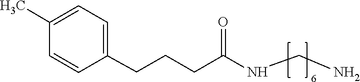 |
| 5 | 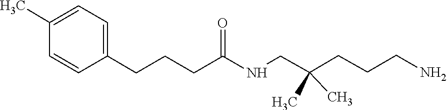 |
| 6 | 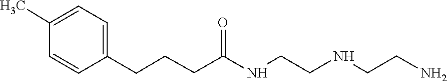 [SMILES: Cc1ccc(CCCC(=O)NCCNCCN)cc1] |
| 7 | 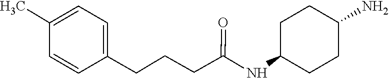 [SMILES: Cc1ccc(CCCC(=O)N[C@H]2CC[C@H](N)CC2)cc1] |
| 8 | 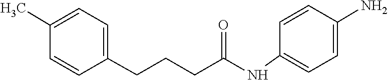 [SMILES: Cc1ccc(CCCC(=O)Nc2ccc(N)cc2)cc1] |
| 9 | 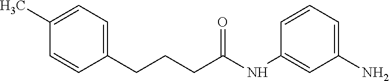 [SMILES: Cc1ccc(CCCC(=O)Nc2cccc(N)c2)cc1] |
| 10 | 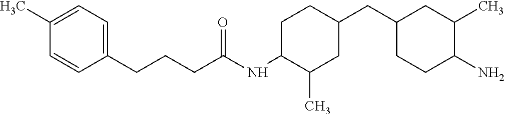 [SMILES: Cc1ccc(CCCC(=O)NC2CCC(CC3CCC(N)C(C)C3)CC2C)cc1] |
| 11 | 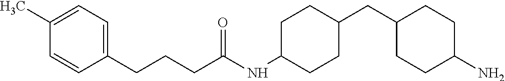 [SMILES: Cc1ccc(CCCC(=O)NC2CCC(CC3CCC(N)CC3)CC2)cc1] |

Where chelator D is a polycarboxylic macrocyclic structure, any molecule of DOTA or DOTAGA as shown in the structure below can be combined with a positive trivalent metal ion:

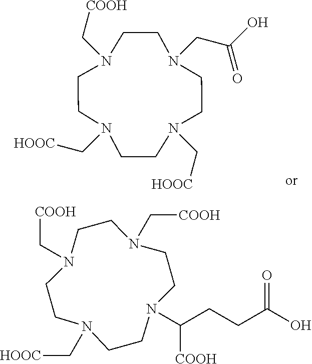

Wherein chelator D structure further comprises 7 types from type D-1 to type D-7, as shown below:

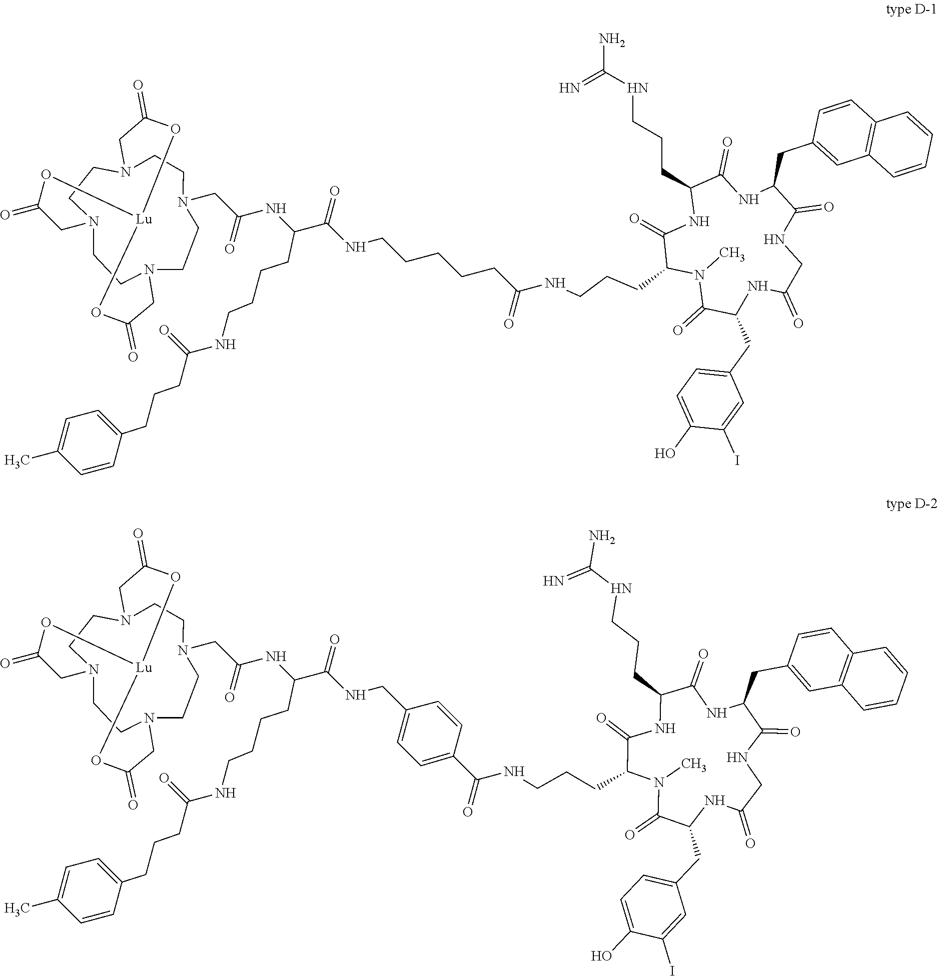

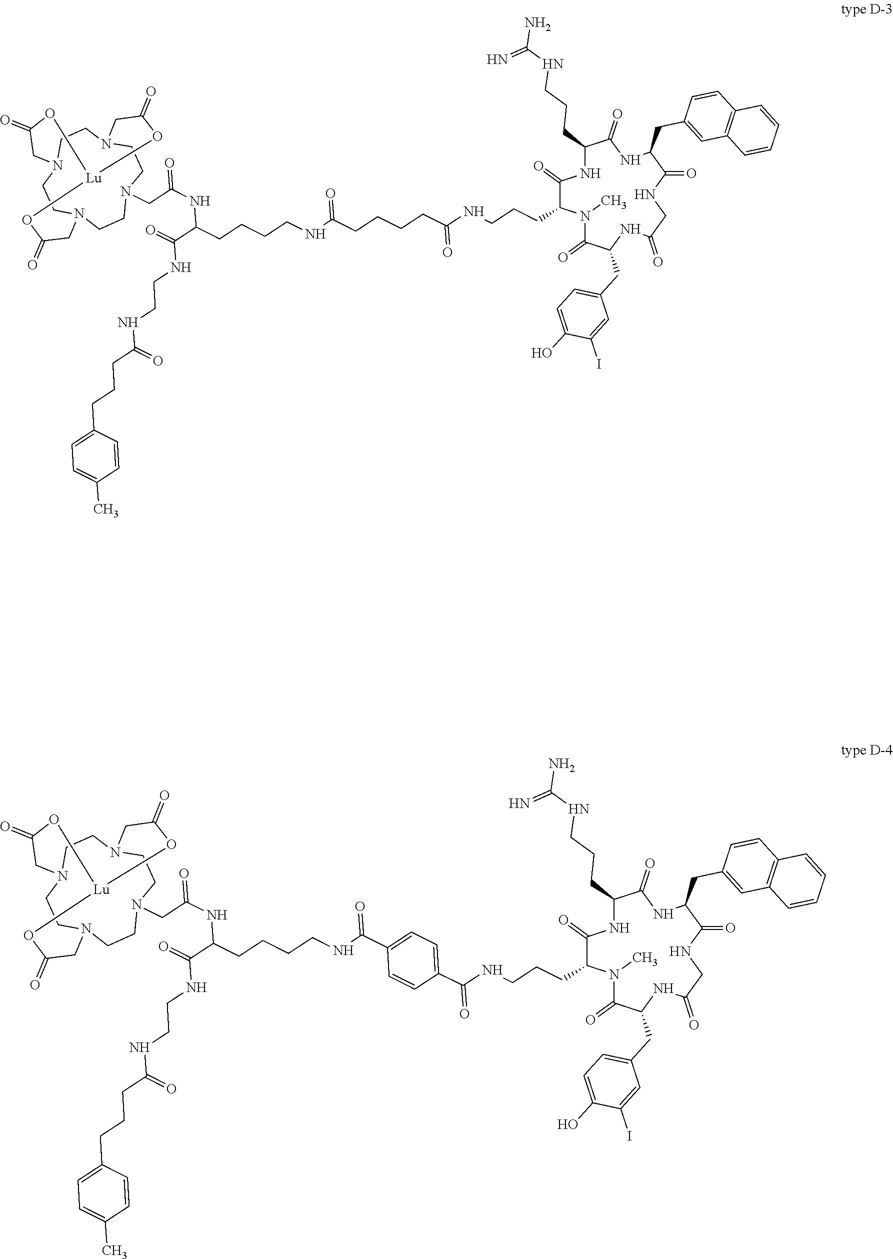

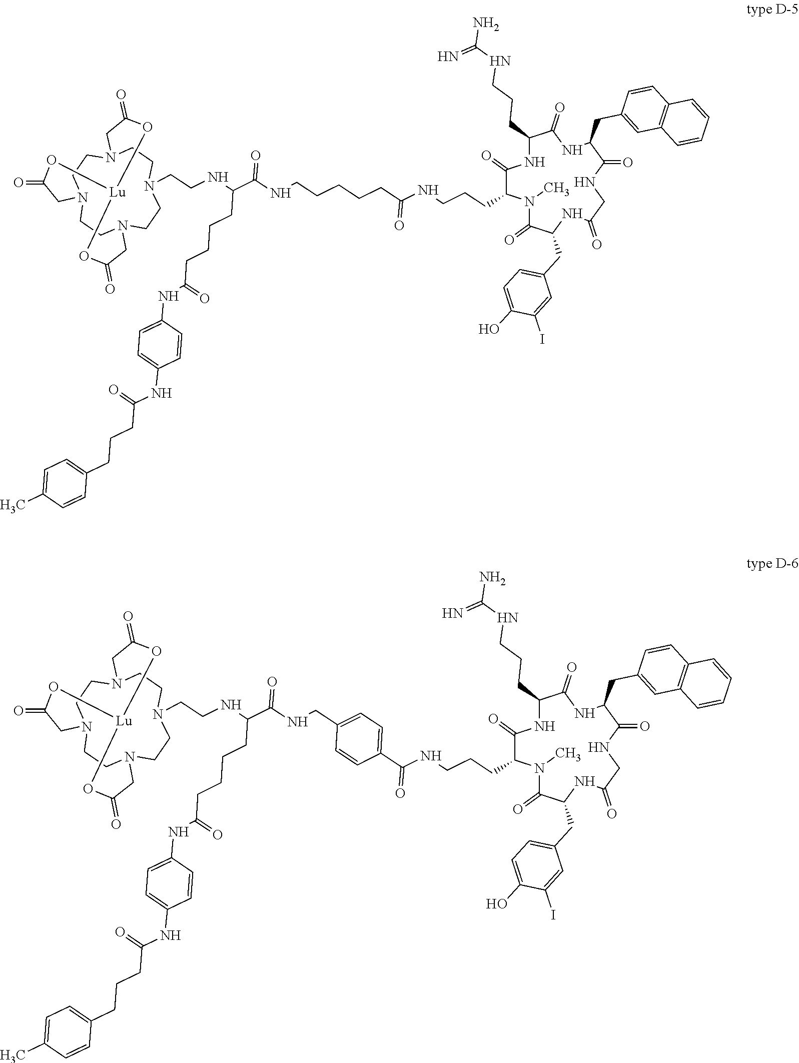

The trivalent metal ion M is selected from one of ¹¹¹In, ⁶⁸Ga, ⁶⁷Ga, ⁹⁰Y or ¹⁷⁷Lu.

### Embodiment 1: Method of Synthesis of D-L2(-B)-L1-A Compound or a Pharmaceutically Acceptable Salt Thereof

Compound I-01(-I-01)-I-17 was synthesized in the following synthesis scheme 1. Lysine (L2-I-01) with two amino groups protected by Fmoc was activated by HATU and DIPEA in the solvent DMF. and to react with 6-amino-hexanoate (L14-17) resin, and then remove the Fmoc protecting group with TFA, H2O and TIPS to generate an intermediate 1-1.

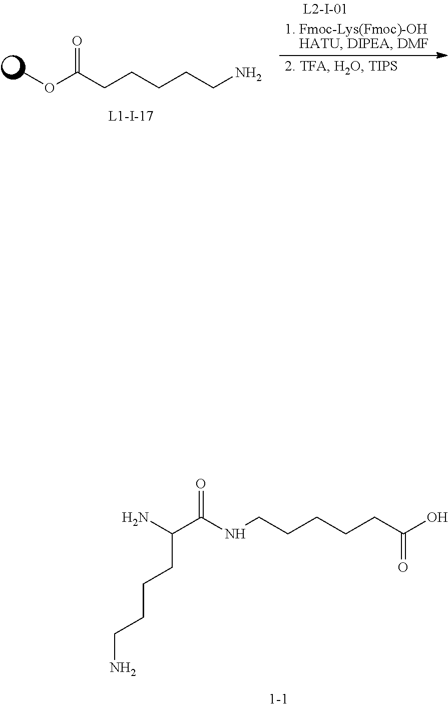

The intermediate 1-1 is still in the solvent DMF to activate the carboxylic acid group via HATU and DIPEA, and react with Orn side chain amino group of A-Pbf (cyclo(D-3-iodo-Tyr-[NMe]-D-Orn-Arg(Pbf)-2-Nal-Gly)) to produce intermediate 1-2.

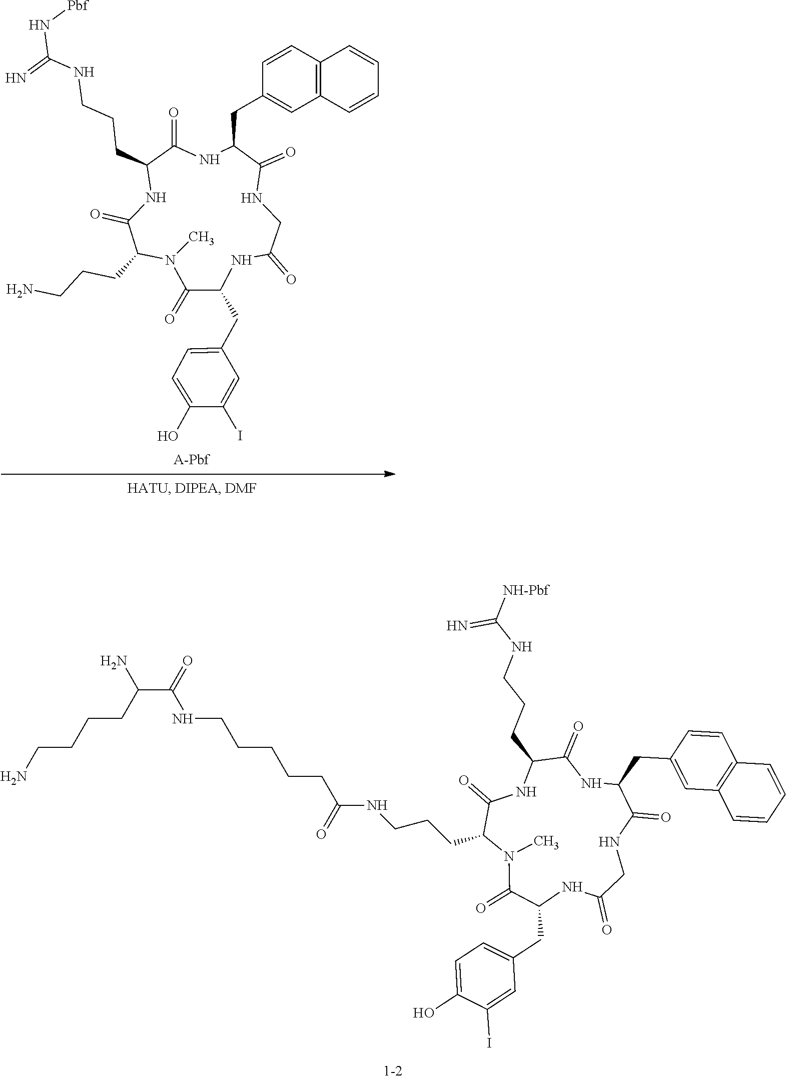

4-methyl-benzenebutanoic acid (B-I-01) activates the carboxylic acid group in solvent DMF via HATU and DIPEA, and reacts with the lysine side chain amino group of intermediate 1-2 to produce intermediate 1-3.

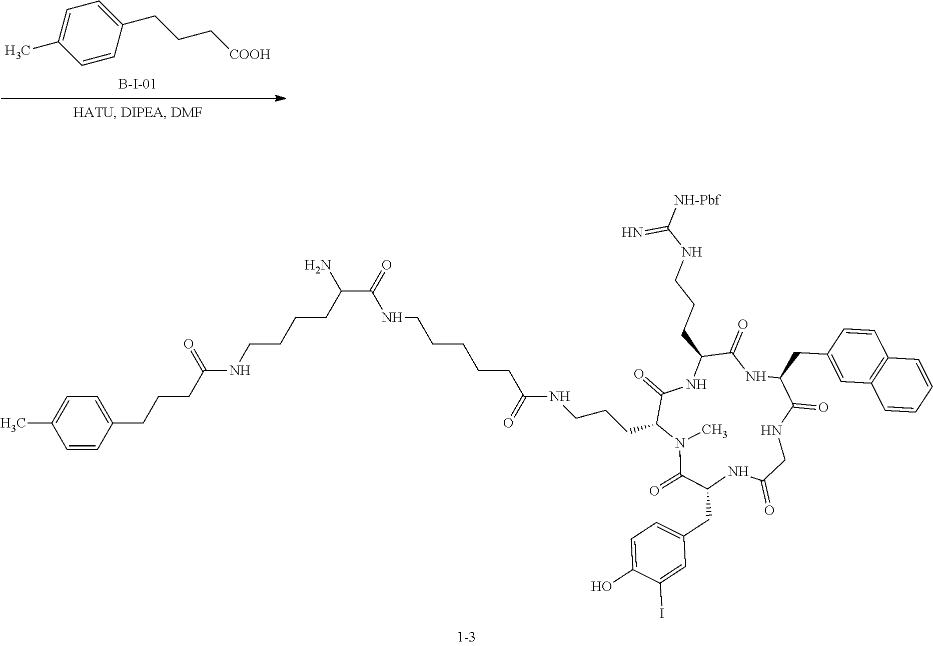

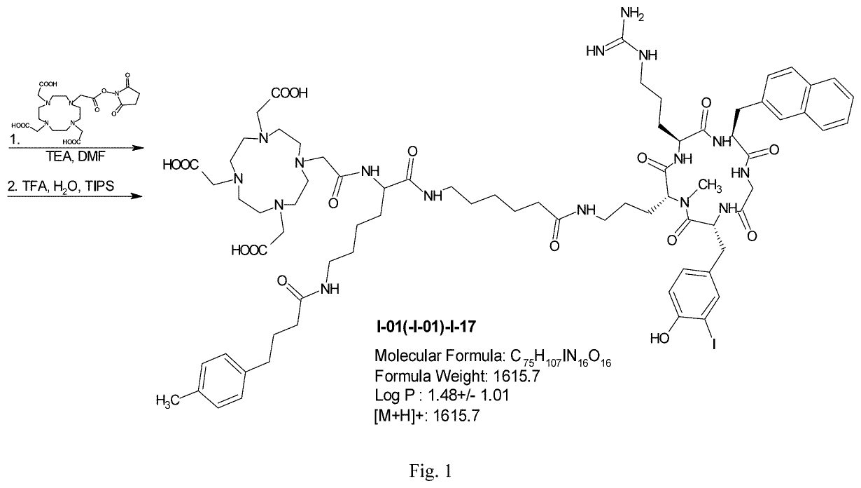

DOTA-NHS hydrolyzes the NHS structure in TEA and DMF solvents to form an activated carboxylic acid group and reacts with the amino group of the lysine in the intermediate 1-3 structure, and then uses TFA, H2O and TIPS to remove the protection base Pbf of the arginine side chain on the A-Pbf structure and generates compound I-01(-I-01)-I-17, the compound molecular formula is C₇₅H₁₀₇IN₁₆O₁₆; the molecular weight is 1615.7 Da; [M+H]+ is 1615.7 Da, as shown in FIG. 1.

The synthesis of compound I-01(-I-01)-I-01 is provided through the following synthesis scheme 1. The lysine (L2-I-01) with two amino groups protected by Fmoc is activated by HATU and DIPEA in the solvent DMF, and reacts with the resin attached with 4-(aminomethyl) benzoate (L14-01), then removes Fmoc protecting group with TFA, H2O and TIPS to produce intermediate 2-1.

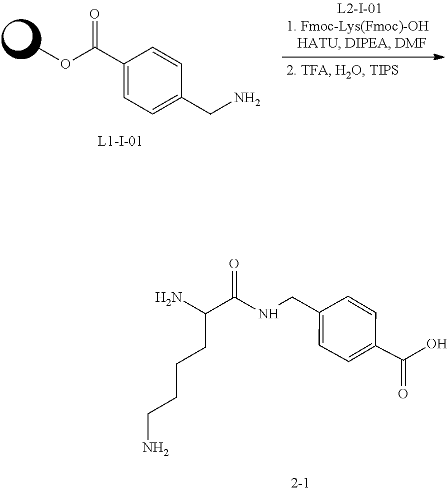 [SMILES: COC(=O)c1ccc(CN)cc1 ; NCCCCC(N)C(=O)NCc1ccc(C(=O)O)cc1]

The intermediate 2-1 is still in the solvent DMF to activate the carboxylic acid group via HATU and DIPEA, and reacts with the Orn side chain amino group A-Pbf to form an intermediate 2-2.

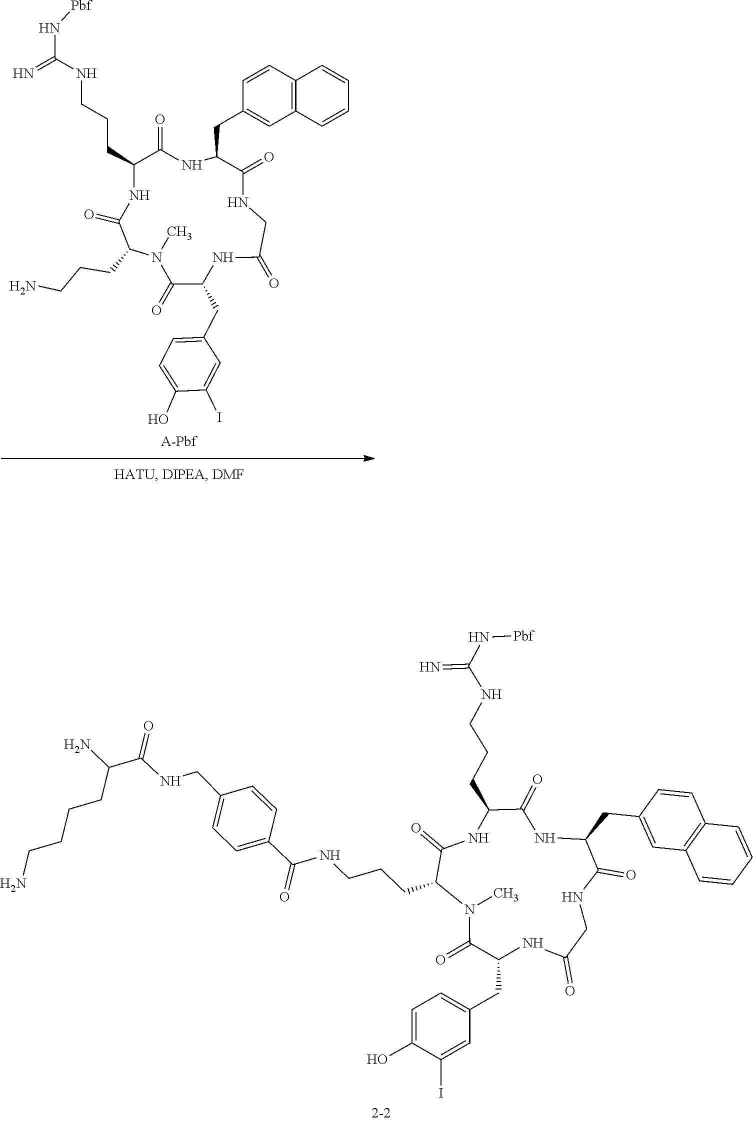

4-methyl-benzenebutanoic acid (B-I-01) activates the carboxylic acid group via HATU and DIPEA in the solvent DMF, and reacts with the lysine side chain amino group of the intermediate 2-2 to form the intermediate 2-3.

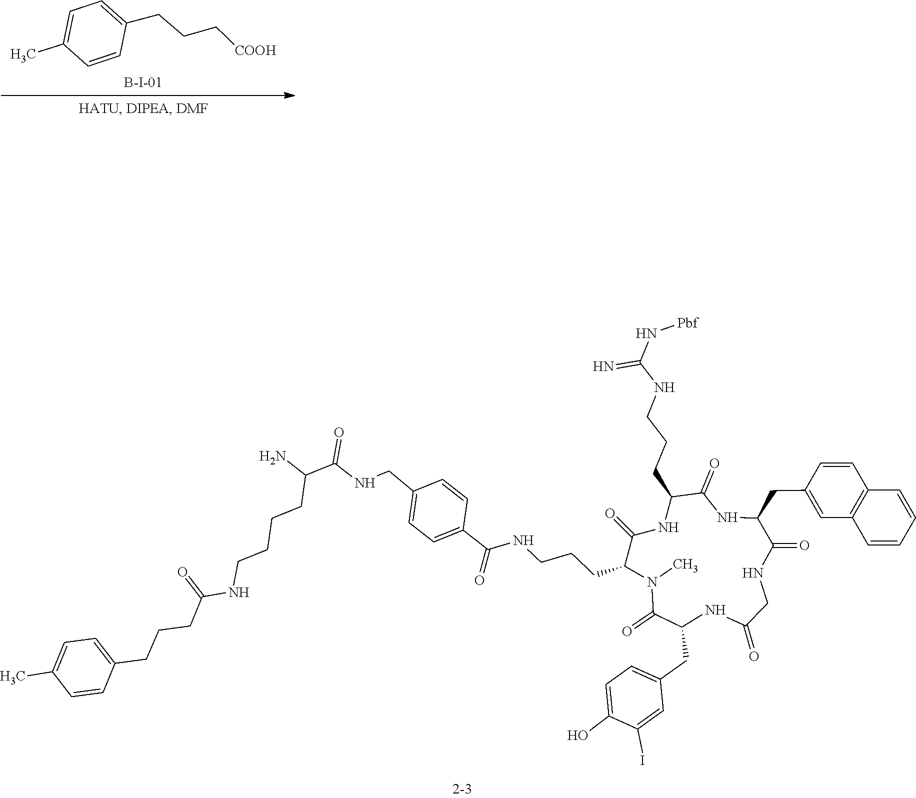

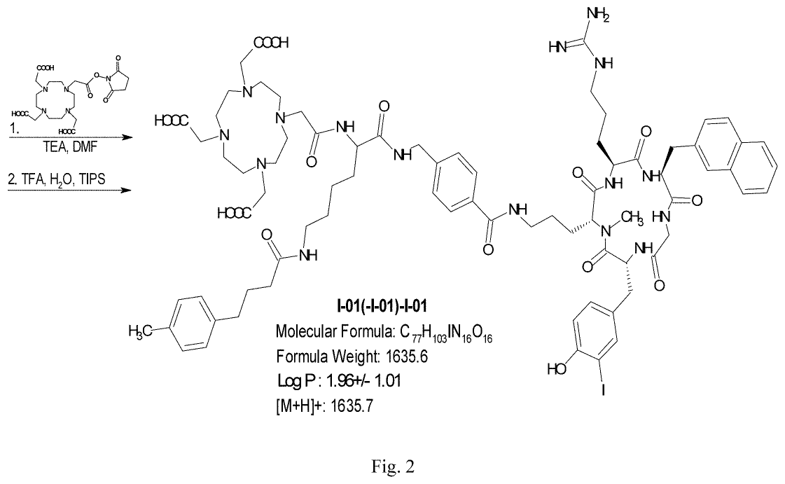

DOTA-NHS hydrolyzes the NHS structure in TEA and DMF solvents to form an activated carboxylic acid group and reacts with the amino group of lysine in the intermediate 2-3 structure, and then uses TFA, H₂O and TIPS to remove the protection Pbf of the arginine side chain on the A-Pbf structure and generates compound I-01(-I-01)-1-01. The compound molecular formula is C₇₇H₁₀₃IN₁₆O₁₆; the molecular weight is 1635.6 Da; [M+H]+ is 1635.7 Da, as shown in FIG. 2. Compound I-01(-II-01)-II-01 is provided through the synthesis scheme 2 shown below. The carboxylic acid group of N6-[(1,1-dimethylethoxy) carbonyl]-lysine (L2-I-01) is activated in the solvent DMF via HATU and DIPEA and reacts with N-(2-aminoethyl)-4-methyl-benzenebutanamide (B-II-01) to produce intermediate 3-1.

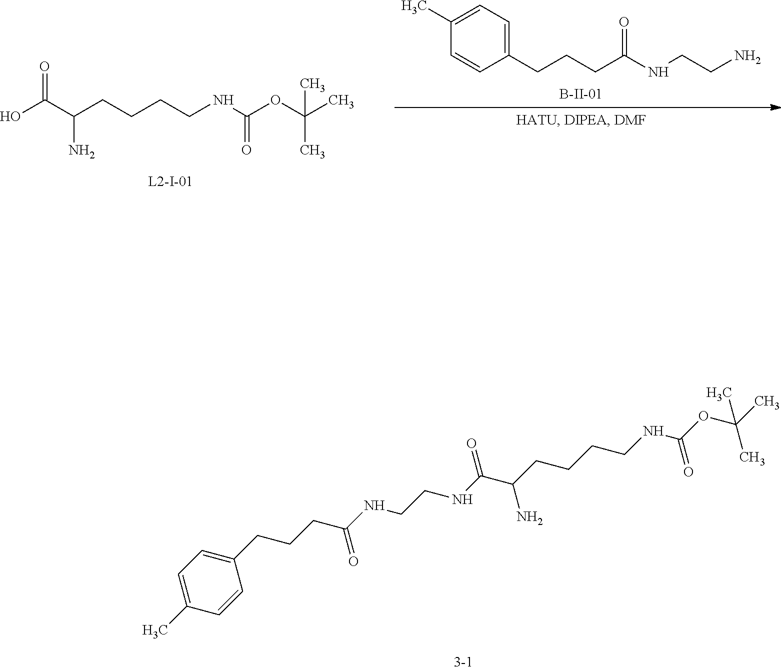 [SMILES: CC(C)(C)OC(=O)NCCCCC(N)C(=O)O ; Cc1ccc(CCCC(=O)NCCN)cc1 ; Cc1ccc(CCCC(=O)NCCNC(=O)C(N)CCCCNC(=O)OC(C)(C)C)cc1]

DOTA-NHS hydrolyzes the NHS structure in TEA and DMF solvents to generate activated carboxylic acid groups and reacts with the unprotected amino group of lysine in the intermediate 3-1 structure, and then removes the protective group t-butylbicarbonate on the amino group with TFA, H₂O and TIPS to produce intermediate 3-2.

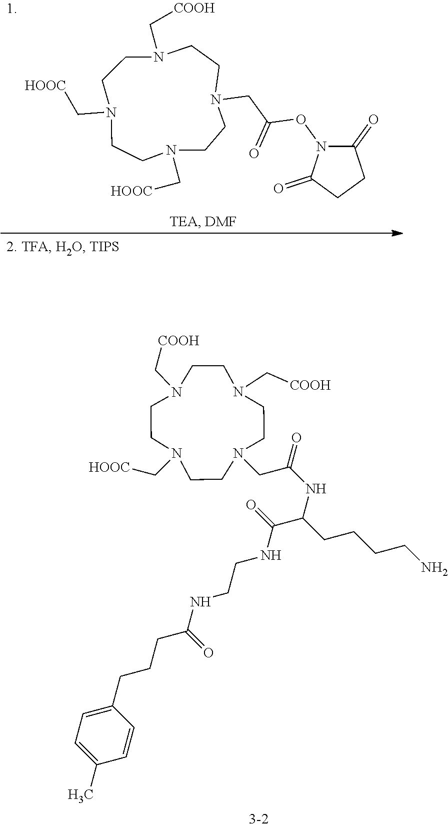 [SMILES: O=C(O)CN1CCN(CC(=O)O)CCN(CC(=O)ON2C(=O)CCC2=O)CCN(CC(=O)O)CC1 ; Cc1ccc(CCCC(=O)NCCNC(=O)C(CCCCN)NC(=O)CN2CCN(CC(=O)O)CCN(CC(=O)O)CCN(CC(=O)O)CC2)cc1]

Hexanedioic acid first activates one end of the carboxylic acid group via HATU and DIPEA in the solvent DMF, reacts with the Orn side chain amino group of A-Pbf, and then uses HCl and H2O to stabilize the activated carboxylic acid group to produce intermediate 3-3.

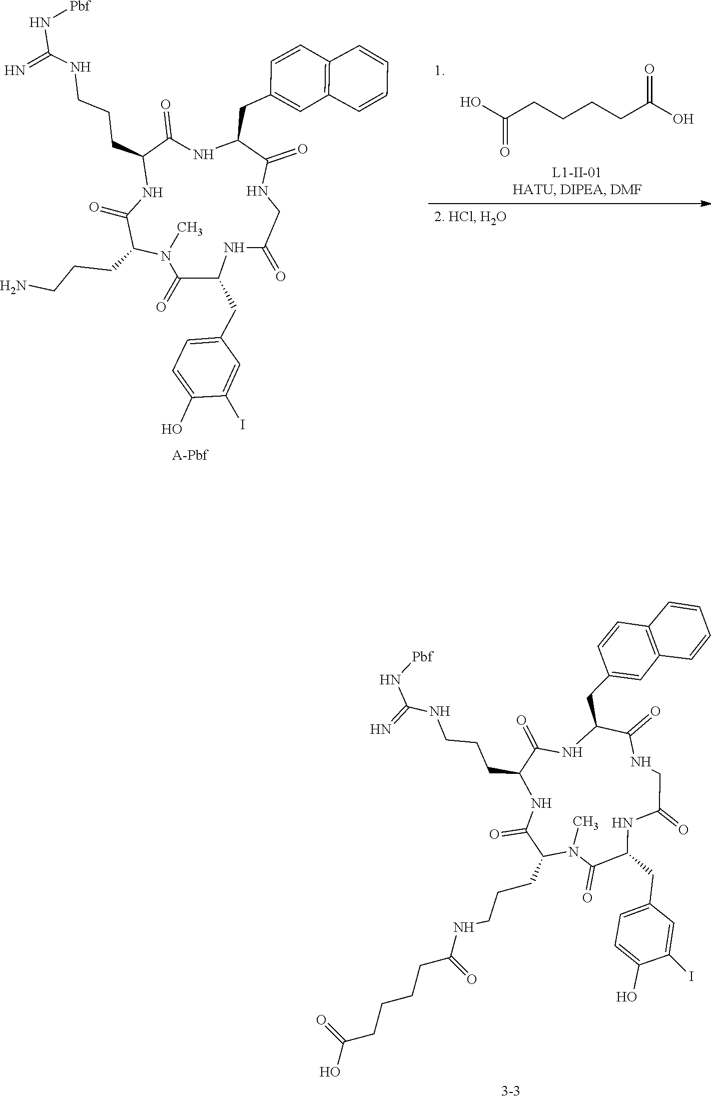

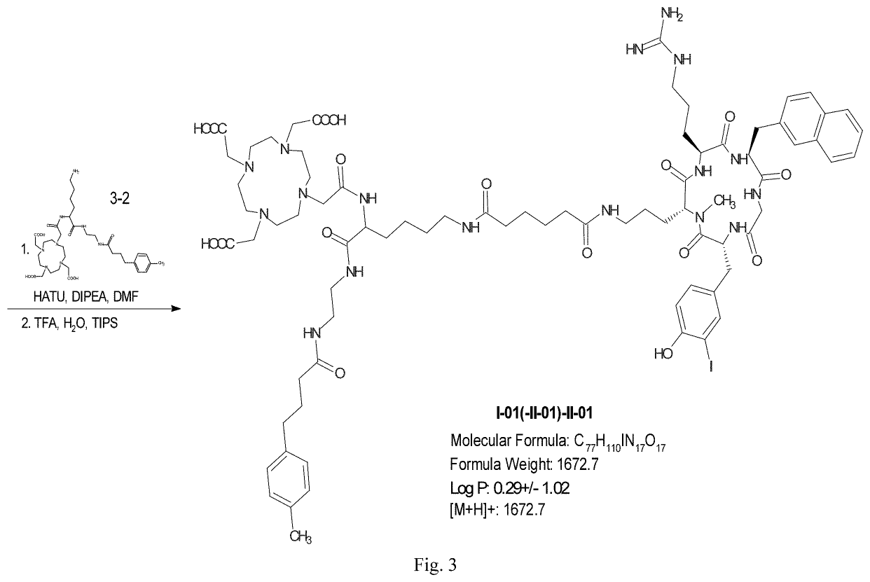

Intermediate 3-3 reacts with the amino group of intermediate 3-2 via HATU and DIPEA activated carboxylic acid group in the solvent DMF, and then removes the protective group Pbf of the arginine side chain on the A-Pbf structure with TFA, H₂O and TIPS to produce the compound I-01(-II-01)-II-01. The compound molecular formula is C₇₇H₁₁₀IN₁₇O₁₇; the molecular weight is 1672.7 Da; [M+H]+ is 1672.7 Da, as shown in FIG. 3. Compound I-01(-II-01)-II-08 is provided through the following synthesis scheme 2 to synthesize N6-[(1,1-dimethylethoxy) carbonyl]-lysine (L2-I-01) in the solvent DMF via HATU and DIPEA to activate the carboxylic acid group and reacts with N-(2-aminoethyl)-4-methyl-benzenebutanamide (B-II-01) to produce intermediate 3-1.

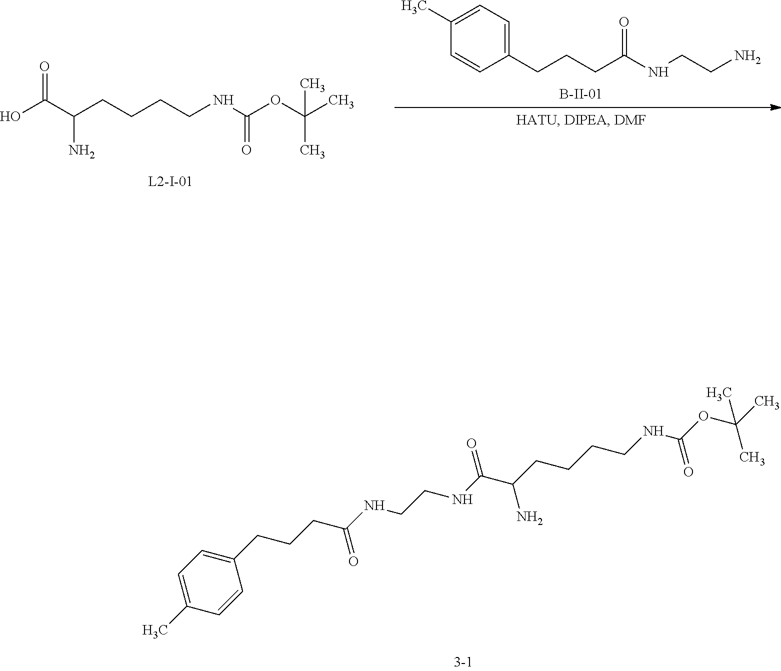 [SMILES: CC(C)(C)OC(=O)NCCCCC(N)C(=O)O ; Cc1ccc(CCCC(=O)NCCN)cc1 ; Cc1ccc(CCCC(=O)NCCNC(=O)C(N)CCCCNC(=O)OC(C)(C)C)cc1]

DOTA-NHS hydrolyzes the NHS structure in TEA and DMF solvents to form an activated carboxylic acid group and reacts with the unprotected amino group of lysine in the intermediate 4-1 structure, and then removes the protective group t-butylbicarbonate on the amino group with TFA, H₂O and TIPS to produce intermediate 3-2.

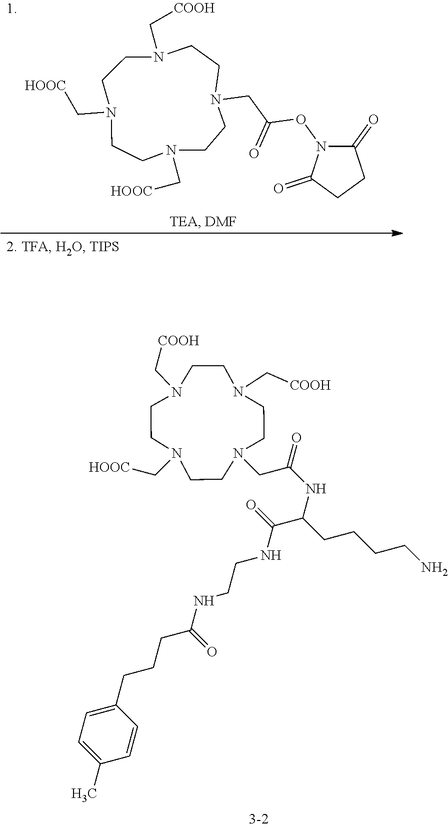 [SMILES: O=C(O)CN1CCN(CC(=O)O)CCN(CC(=O)ON2C(=O)CCC2=O)CCN(CC(=O)O)CC1 ; Cc1ccc(CCCC(=O)NCCNC(=O)C(CCCCN)NC(=O)CN2CCN(CC(=O)O)CCN(CC(=O)O)CCN(CC(=O)O)CC2)cc1]

1,4-Benzenedicarboxylic acid first activates one end of the carboxylic acid group via HATU and DIPEA in the solvent DMF, reacts with the Orn side chain amino group of A-Pbf, and then uses HCl and H₂O to stabilize the activated carboxylic acid group to form an intermediate 4-1.

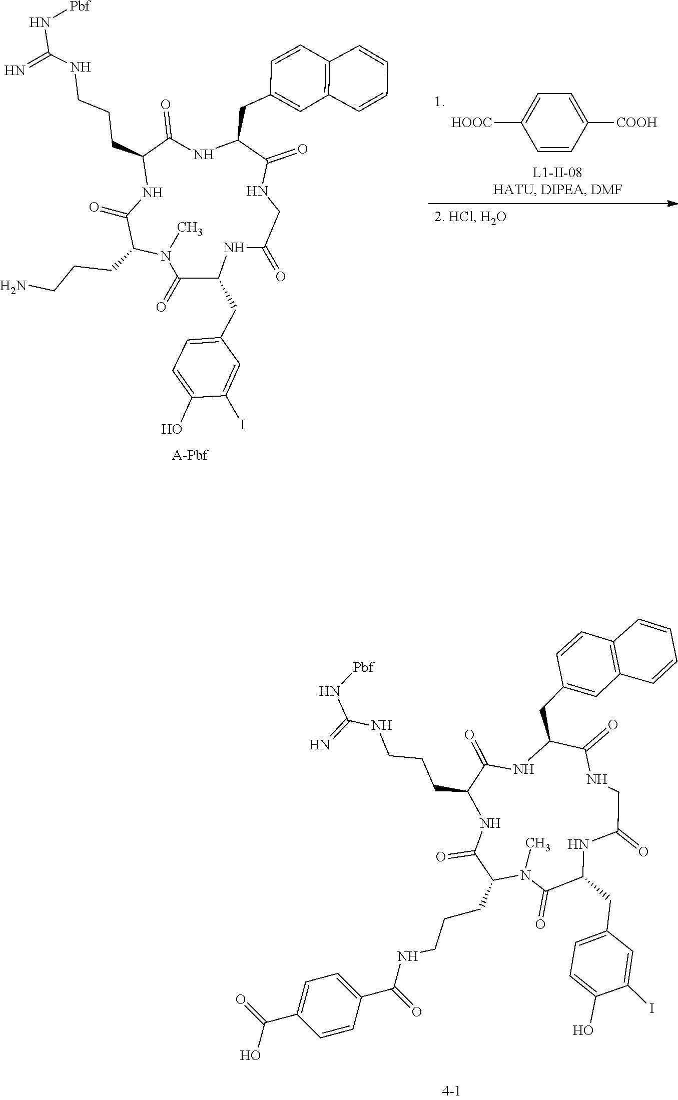

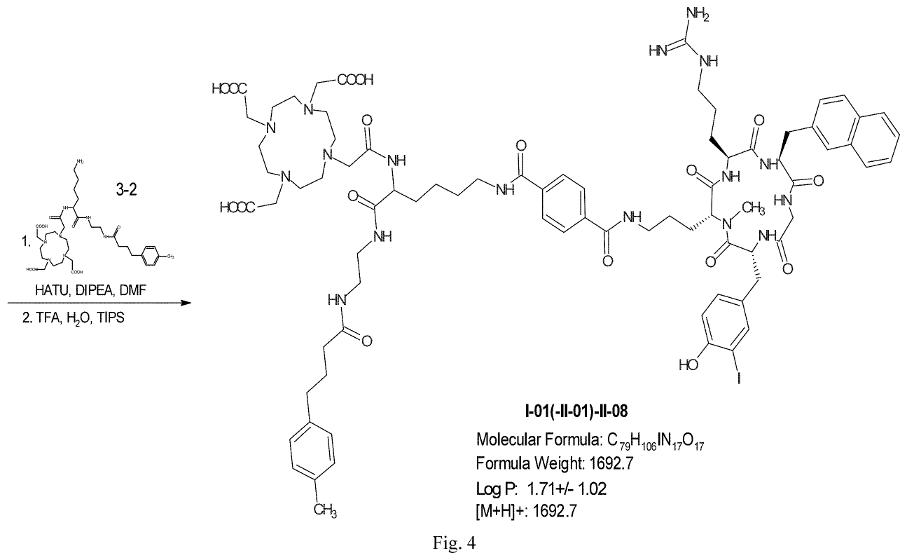

Intermediate 4-1 reacts with the amino group of intermediate 3-2 via HATU and DIPEA activated carboxylic acid group in the solvent DMF, and then removes the protective group Pbf of the arginine side chain on the A-Pbf structure with TFA, H₂O and TIPS to generate the compound I-01(-II-01)-II-08. The compound molecular formula is C₇₉H₁₀₆IN₁₇O₁₇; the molecular weight is 1692.7 Da; [M+H]+ is 1692.7 Da, as shown in FIG. 4. Compound II-03(-II-08)-I-17 is provided through the following synthesis scheme 3 to synthesize 6-Aminohexanoic acid (L1-I-17) in the solvent DMF via HATU and DIPEA to activate the carboxylic acid group, and reacts with A-Pbfs Orn side chain amino group to produce intermediate 5-1.

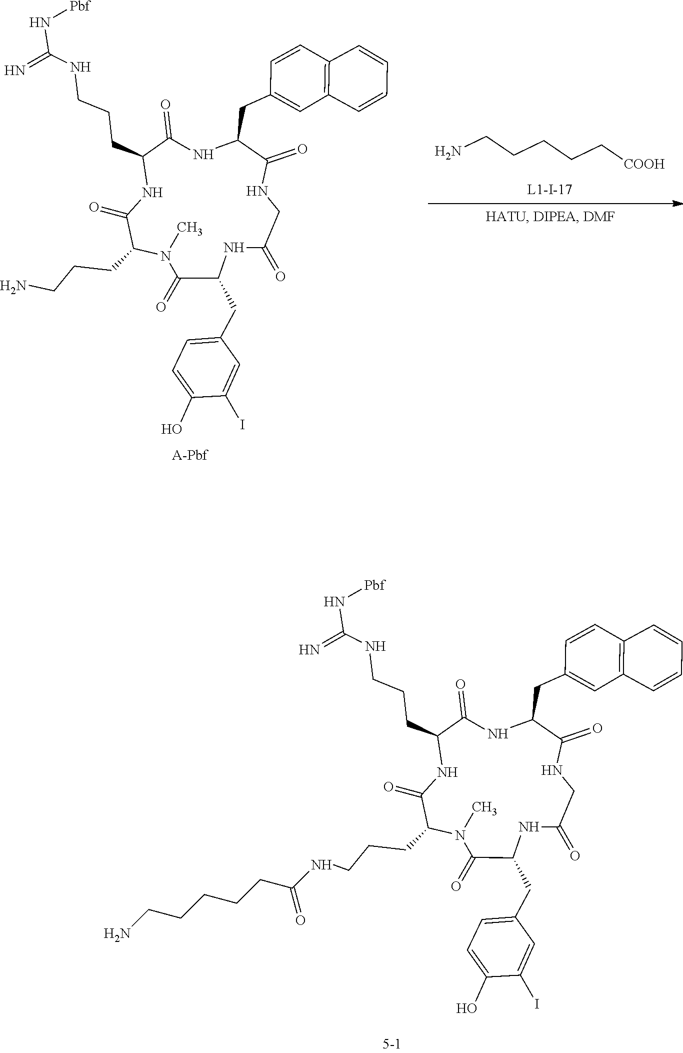

(2S)-2-(9H-Fluoren-9-ylmethoxycarbonylamino)-7-[(2-methylpropan-2-yl)oxy]-7-oxoheptanedioic acid (L2-II-03) via HATU and DIPEA in solvent DMF to activate the carboxylic acid group and reacts with the intermediate 5-1 and then removes the protective group Pbf of the arginine side chain of the A-Pbf structure and the protective group Fmoc and tBu of L2-II-03 with TFA, H₂O and TIPS and produces intermediate 5-2.

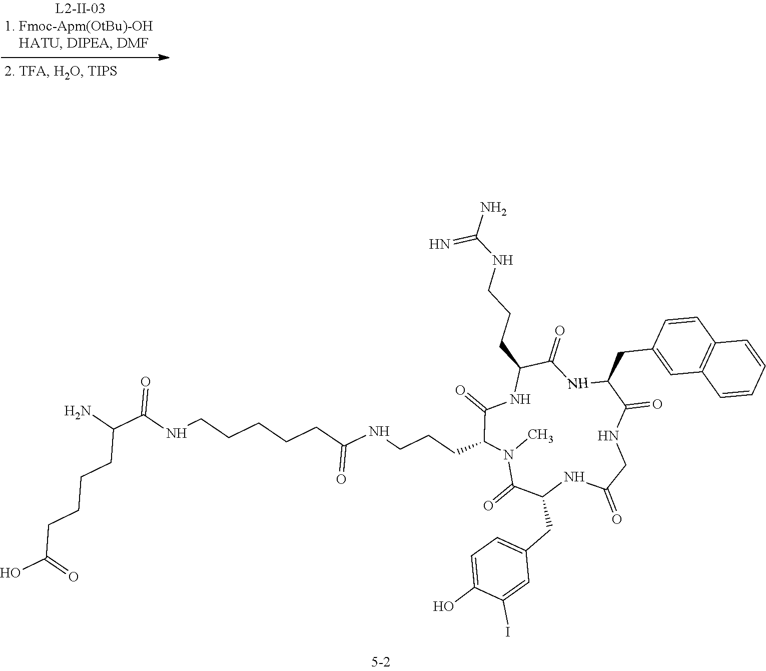 [SMILES: CN1C(=O)[C@@H](Cc2ccc(O)c(I)c2)NC(=O)CNC(=O)[C@H](Cc2ccc3ccccc3c2)NC(=O)[C@H](CCCNC(=N)N)NC(=O)[C@H]1CCCNC(=O)CCCCCNC(=O)C(N)CCCCC(=O)O]

1,4,7,10-Tetraazacyclododecane-1,4,7,10-tetraacetic acid, 1,4,7-tris(1,1-dimethylethyl) ester activates the carboxylic acid group via HATU and DIPEA in the solvent DMF, and reacts with the amino group of L2-II-03 in intermediate 5-2 to produce intermediate 5-3.

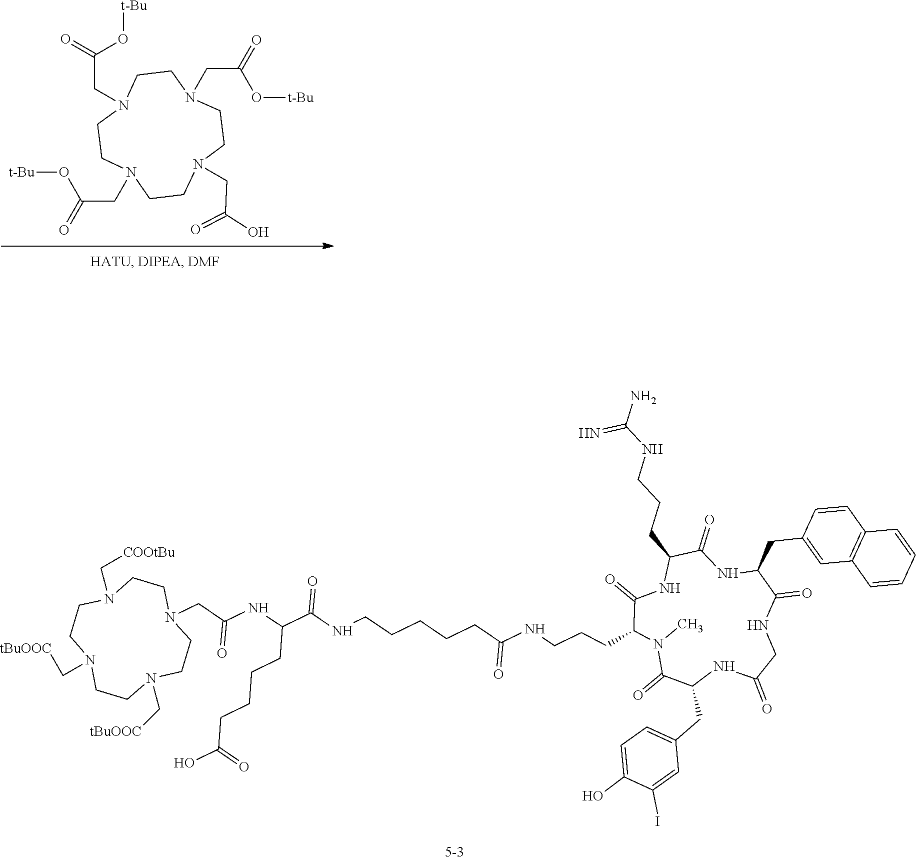

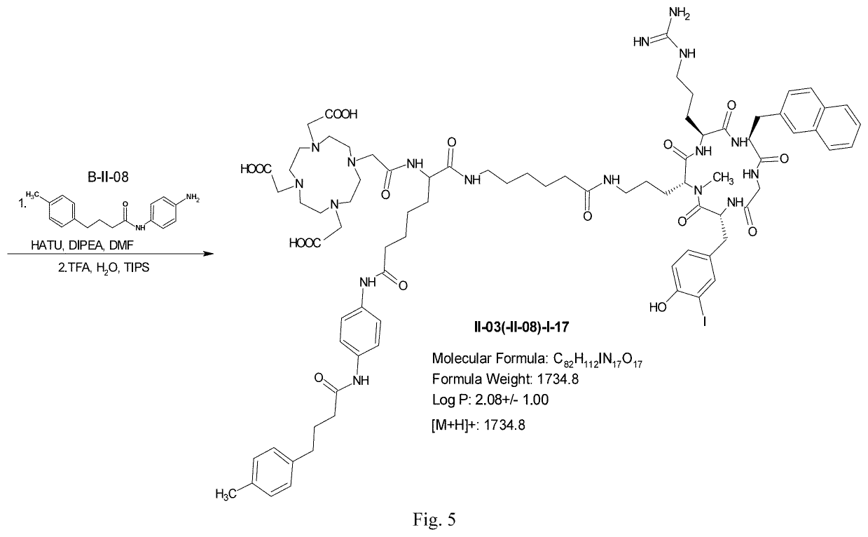

Intermediate 5-3 activates the carboxylic acid group of L2-II-03 in the structure via HATU and DIPEA in the solvent DMF, reacts with N-(-I-aminophenyl)-4-methyl-benzenebutanamide (B-II-08), and then uses TFA, H₂O and TIPS to remove the protective group tBu on 1,4,7,10-Tetraazacyclododecane-1,4,7,10-tetraacetic acid, 1,4,7-tris(1,1-dimethylethyl) ester to generate the compound II-03(-II-08)-I-17. The compound molecular formula is C₈₂H₁₁₂IN₁₇O₁₇; the molecular weight is 1734.8 Da; [M+H]+ is 1734.8 Da, as shown in FIG. 5.

Compound II-01(-II-01)-I-01 is provided through the following synthesis scheme 4 to synthesize 4-(Aminomethyl)-benzoic acid (L14-01) in the solvent DMF via HATU and DIPEA to activate the carboxylic acid group, and reacts with the Orn side chain amino group of A-Pbf to produce intermediate 6-1.

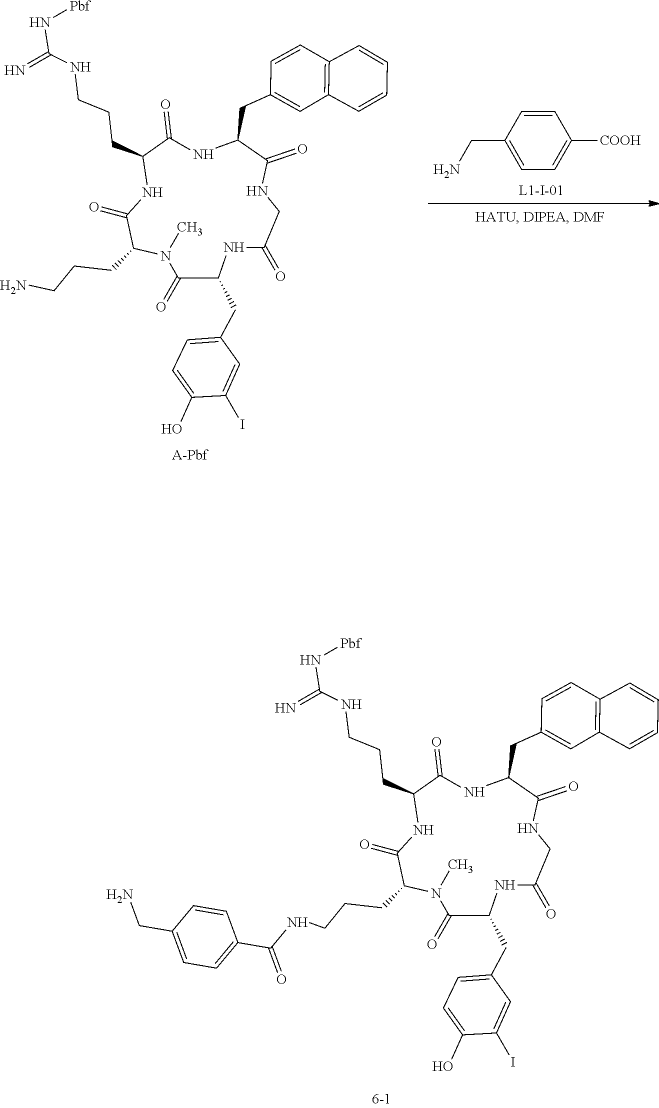

N-[(9H-fluoren-9-ylmethoxy)carbonyl]-, 5-(1,1-dimethylethyl) ester glutamic acid (L2-II-01) activates the carboxylic acid group via HATU and DIPEA in the solvent DMF and reacts with the intermediate 6-1, and then uses TFA, H₂O and TIPS to remove the protective group Pbf of the arginine side chain on the A-Pbf structure and the protective group Fmoc and tBu of L2-II-01 to produce the intermediate 6-2.

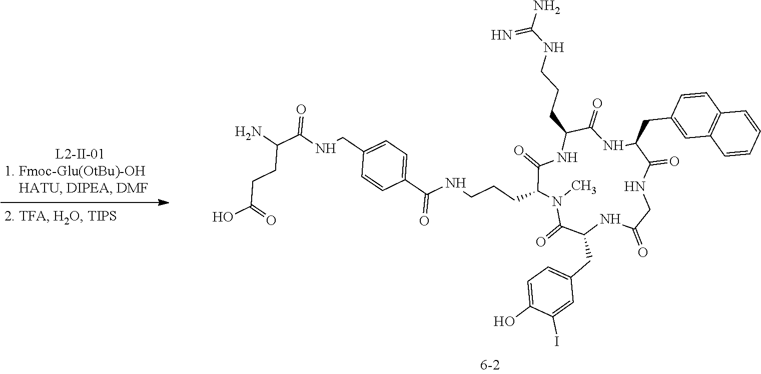 [SMILES: CN1C(=O)[C@@H](Cc2ccc(O)c(I)c2)NC(=O)CNC(=O)[C@H](Cc2ccc3ccccc3c2)NC(=O)[C@H](CCCNC(=N)N)NC(=O)[C@H]1CCCNC(=O)c1ccc(CNC(=O)C(N)CCC(=O)O)cc1]

1,4,7,10-Tetraazacyclododecane-1,4,7,10-tetraacetic acid, 1,4,7-tris(1,1-dimethylethyl) ester activates the carboxylic acid group in the solvent DMF via HATU and DIPEA, and reacts with the amino group of L2-II-01 in intermediate 6-2 to produce intermediate 6-3.

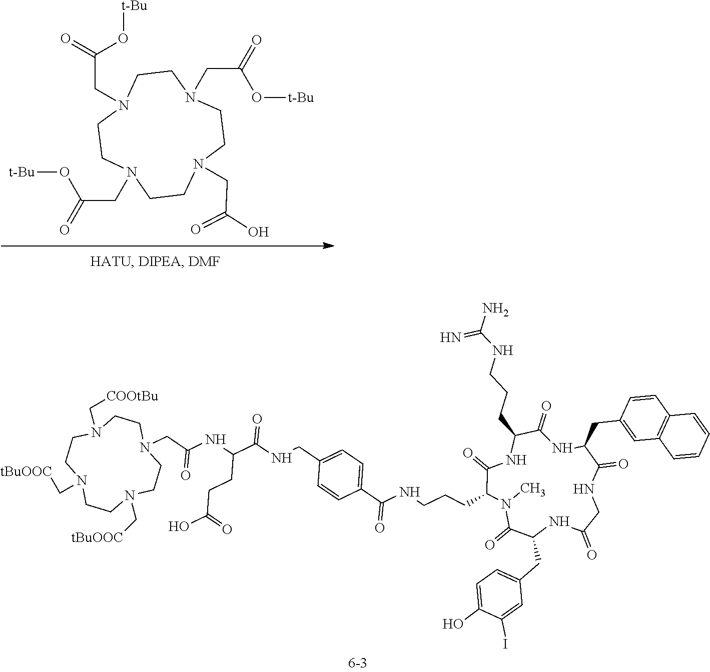

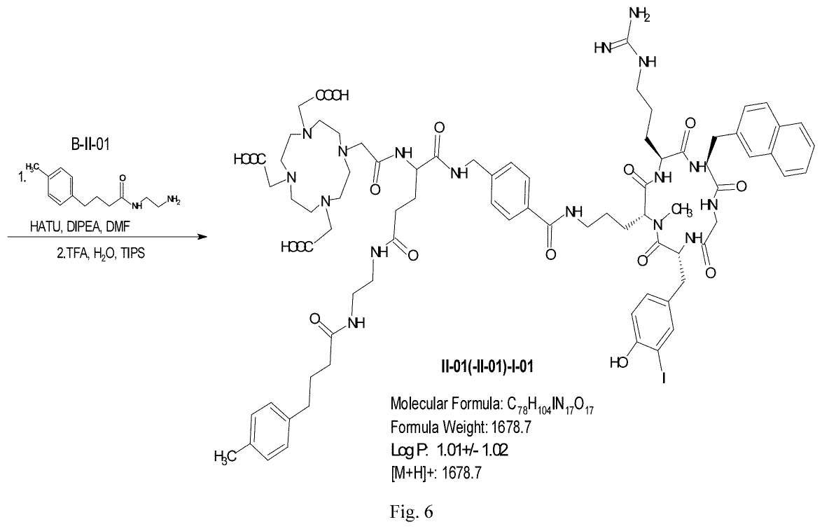

The intermediate 6-3 activates the carboxylic acid group of L2-II-01 in the structure in the solvent DMF via HATU and DIPEA, reacts with N-(2-aminoethyl)-4-methyl-benzenebutanamide (B-II-01), and then use TFA, H₂O and TIPS to remove the protective group tBu on 1,4,7,10-Tetraazacyclododecane-1,4,7,10-tetraacetic acid, 1,4,7-tris(1,1-dimethylethyl) ester to produce the compound II-01(-II-01)-I-01. The molecular formula of the compound is C₇₈H₁₀₄IN₁₇O₁₇; the molecular weight is 1678.7 Da; [M+H]+ is 1678.7 Da, as shown in FIG. 6.

### Embodiment 2: Radioactive labeling of D-L2(-B)-L1-A compound

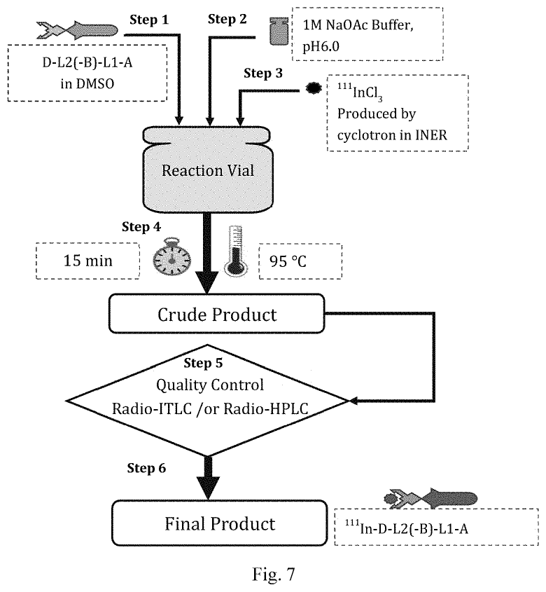

D-L2(-B)-L1-A compound I-01(-I-01)-I-17 or I-01(-I-01)-I-01 is formulated with dimethyl sulfoxide (DMSO) to produce 20 mg/mL. In-111 radionuclide labeling method is to add 20 μg I-01(-I-01)-I-17 or I-01(-I-01)-I-01 compound to 1.0 M sodium acetate buffer solution pH 6.0 (NaOAc), and after ultrasonic vibration add in activity 6.20-6.27 mCi In-111 and mix well, put in 95° C. precision thermostat temperature controller for reaction for 15 minutes, and vibrate at 500 rpm during heating. After the reaction completed and cooled, take a sample for analyzing the labeling efficiency and radiochemical purity with radio-ITLC or radio-HPLC, as shown in FIG. 7.

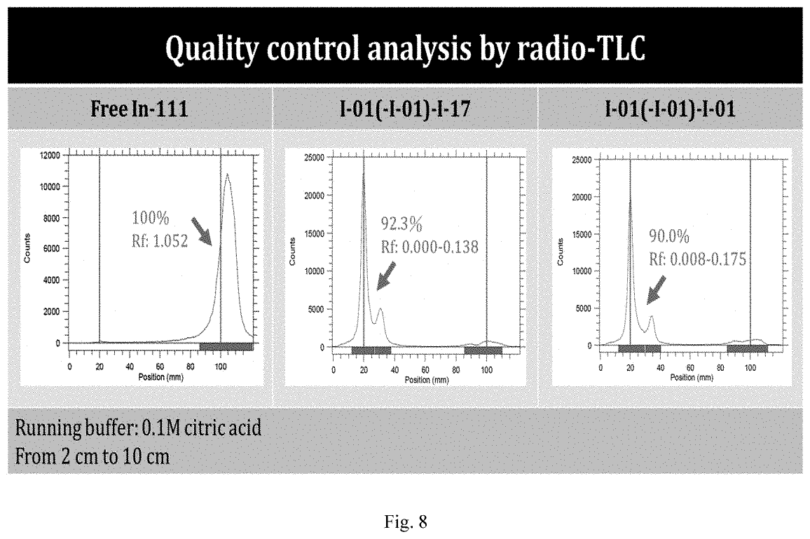

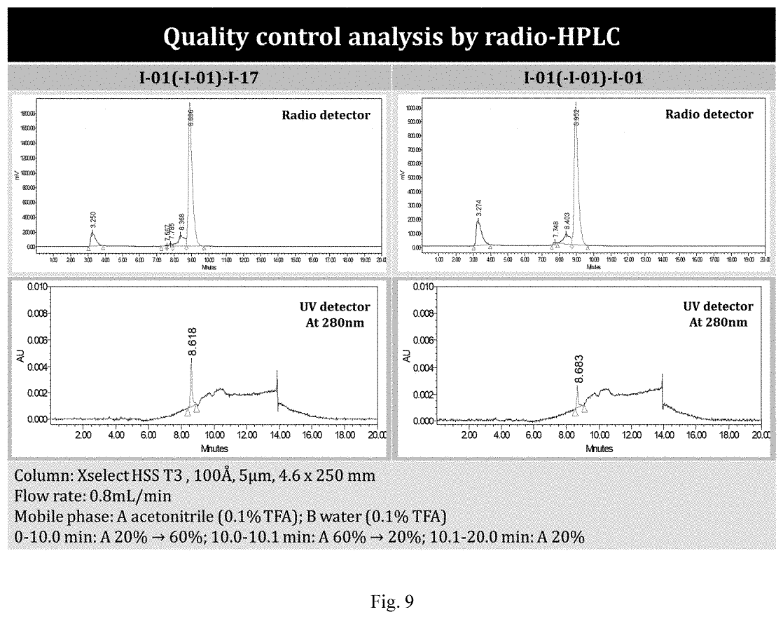

In the Radio-TLC (radio-thin layer chromatography), the developing solution for analysis is 0.1M citric acid, and the Rf (rate of flow) of the compound with reacted In-111 is 0.000-0.138, and the Rf of the compound without reacted In-111 is 0.008-0.175, as shown in FIG. 8. In the radio-HPLC (radio-high pressure liquid chromatography), the analysis mobile phase is A and B, and A is acetonitrile containing 0.1% TFA, and B is deionized water containing 0.1% TFA; the stationary phase includes XSelect HSS T3 column (5 μm, 4.6 mm×250 mm), speed 0.8 mL/min, analysis for 20 minutes, and use of the gradient elution: 0-10 minutes A is 20% to 90%, 10-10.1 minutes A is 90% to 20%, and 10.1-20 minutes A is 20%. After 15 minutes of reaction, the radiochemical purity of the radioactive compound can be greater than 90%, as shown in FIG. 9.

Embodiment 3: Radionuclide Labeled D-L2(-B)-L1-A is Used for Animal In Vivo imaging PC-3 human frontal adenocarcinoma cells were inoculated into the forelimbs of SCID mice at 4×10⁶, and about 3 weeks after inoculation, the radionuclide In-111 labeled D-L2(-B)-L1-A was used to adjust with injection water, and 469-527 μCi of radioactive drugs were intravenously injected into the tail vein, and nano SPECT/CT imaging was performed after administration at 1, 4, 24, 48, 72, and 96 hours, respectively. Nano SPECT/CT was used to track the distribution of radiopharmaceuticals in the animal body at each of the time points after administration, and analysis software was used to enter the image into the semi-definite image to obtain the SPECT value from the test substance with known activity at each of the time points. The linear ratio formula was derived between the SPECT calculated value and the actual activity, and the SPECT calculated value was then used at each target organ circled area to obtain the live/volume of each target organ with interpolation or extrapolation linear formula.

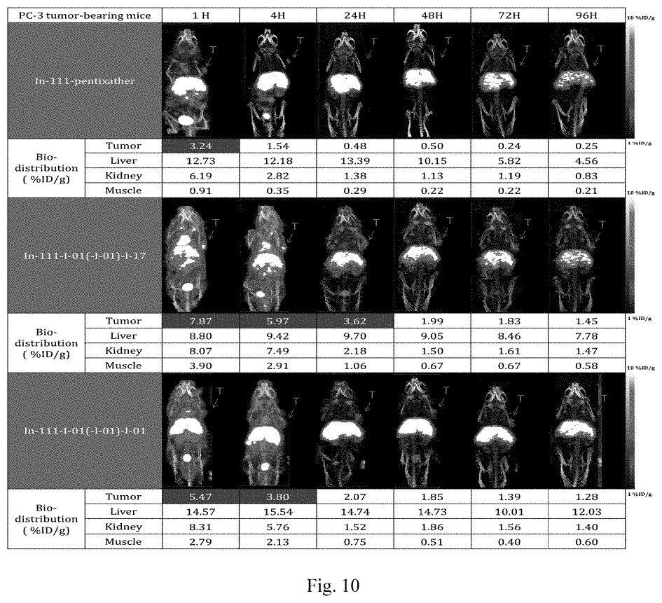

For performing the calculation we assume that every 1 mL of injection dosage is 1 g, and the circled area in the mouse body is 1 g per 1 cm³. Furthermore, since the activity volume of the administered injection and the weight of the experimental mouse are known, each gram of In-111-D-L2(-B)-L1-A's biological distribution ratio (% ID/g) in each organ can be deduced, and will focus on organ tumor, liver, kidney and muscle into the distribution comparison and obtain ratio, as shown in FIG. 10.

What is claimed is:

1. A compound of formula D-L2(-B)-L1-A or a pharmaceutically acceptable salt thereof, wherein the structure A of the formula is cyclo (D-3-iodo-Tyr-[NMe]-D-Orn(-L1-)-Arg-2-Nal-Gly), and an amide bond is formed by a branched amino group (—NH2) of Orn molecule and a carboxylic acid group (—COOH) of L1, wherein the L1 structure has a two-end connection function including type L1-I and type L1-II, wherein the L2 structure has a three-end connection function including type L2-I and type L2-II, wherein the compound of formula D-L2(-B)-L1-A or a pharmaceutically acceptable salt thereof comprises CXCR4 binding active structure A, albumin binding active structure B and radioactive chelator structure D;
wherein the type L1-I provides a carboxylic acid group at one end, and provides an amino group at the other end, wherein the carboxylic acid group end is connected to the structure A to form an amide bond, wherein the amino group end is connected to any molecule of L2-I or L2-II structure to form an amide bond, and wherein the L1-I is selected from one of the group consisting of 1 to 26 molecular structures listed in Table 1;
wherein the Type L1-II provides a carboxylic acid groups at both ends, one end of the carboxylic acid group and the structure A form an amide bond, and the other end of the carboxylic acid group and any molecule of
the type L2-I form an amide bond, and wherein the L1-II is selected from one of the group consisting of 1 to 9 molecular structures listed in Table 2;
wherein the type L2-I provides an α-carboxylate, and α-amino and a branched amino molecule, wherein the α-amino group and a chelator structure D form an amide bond, and the branched amino group and the structure B-I form an amide bond, the α-carboxylic acid group end and any structure of L1-I form amide bond; wherein the α-amino group and structure D form an amide bond, and the branched amino group and any structure of L1-II form a amide bond, and the α-carboxylic acid group end and structure B-II form amide bond; wherein the branched amino group and structure D form an amide bond, and the α-amino group and any structure of L1-II form an amide bond, and the α-carboxylic acid group end and the structure B—II form an amide bond, and wherein L2-I is selected from one of the group consisting of 1 to 3 molecular structures listed in Table 3;
wherein L2-II provides an α-carboxylic acid group, a branched carboxylic acid group and α-amino molecules, wherein the α-amino end and structure D form an amide bond, the α-carboxylic acid group end and the B-II form an amide bond, and the branched carboxylic acid group end forms an amide bond with any structure of L1-I; wherein the α-amino end and the D structure form an amide bond, the branched carboxylic acid group end and the B-II form an amide bond, and the α-carboxylic acid group end forms an amide bond with any structure of L1-I, and wherein the L2-II is selected from one of the group consisting of 1 to 4 molecular structures listed in Table 4;
wherein the B structure having type B-I and type B-II is connected with L2, wherein a carboxylic acid group and the L2-I of the B-I structure form an amide bond, and wherein the B-I structure is selected from the group consisting of 1 to 12 molecular structures listed in Table 5;
wherein B-II having an amino group to form an amide bond with L2-II, and B—II is selected from one of the group consisting of molecular structures 1 to 11 listed in Table 6; and
wherein the chelator D is a polycarboxylic macrocyclic structure DOTA or DOTAGA capable of binding with a positive trivalent metal ion M, and the trivalent metal ion M is selected from ¹¹¹In, ⁶⁸Ga, ⁶⁷Ga, ⁹⁰Y or ¹⁷⁷Lu.

2. The compound of formula D-L2(-B)-L1-A or a pharmaceutically acceptable salt of claim 1, wherein A is cyclo (D-3-iodo-Tyr-[NMe]-D-Orn(-L1-)-Arg-2-Nal-Gly), L2 is lysine (L2-I-1), and D is DOTA.

3. The compound of formula D-L2(-B)-L1-A or a pharmaceutically acceptable salt thereof of claim 2, wherein the structure is shown below: 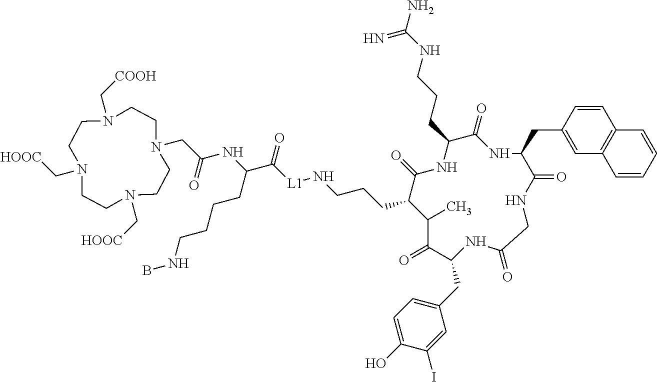
wherein the L1 structure is type L1-I, one end forms a carboxylic acid group, and the other end forms an amino group, and the carboxylic acid group end forms an amide bond with structure A, wherein L1-I is selected from the group consisting of molecular structures 1 to 26 listed in Table 1;
wherein the L1 structure is type L1-II having two ends with a carboxylic acid group, one end of the carboxylic acid group forms an amide bond with structure A, and the L1-II structure is selected from the group consisting of 1 to 9 molecular structures listed in Table 2; and
wherein the B structure is type B-I having a carboxylic acid group, and the B-1 structure is selected from the group consisting of molecular structures 1 to 12 listed in Table 5.

4. The compound of formula D-L2(-B)-L1-A or a pharmaceutically acceptable salt thereof of claim 1, wherein A is cyclo (D-3-iodo-Tyr-[NMe]-D-Orn(-L1-)-Arg-2-Nal-Gly), L2 is 2-aminoheptanedioic acid (L2-11-3), and D is DOTA.

5. The compound of formula D-L2(-B)-L1-A or a pharmaceutically acceptable salt thereof of claim 4, wherein the structure of the compound of formula D-L2(-B)-L1-A or a pharmaceutically acceptable salt thereof is shown below: 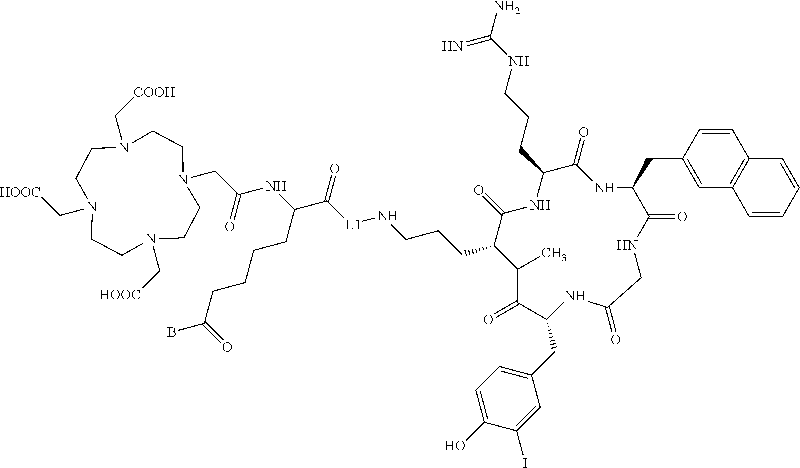
wherein the L1 structure is L1-I, a carboxylic acid group is provided at one end, and an amino group is provided at the other end, and the carboxylic acid group end and structure A form an amide bond, wherein the L1-I is selected from one of the group consisting of molecular structures 1 to 26 listed in Table 1;
wherein the structure B is of type B-II having an amino group, and is selected from one of the group consisting of molecular structures 1 to 11 listed in Table 6.

6. The compound of formula D-L2(-B)-L1-A or a pharmaceutically acceptable salt thereof of claim 1, wherein the structure A is cyclo (D-3-iodo-Tyr-[NMe]-D-Orn(-L1-)-Arg-2-Nal-Gly), L2 is glutamic acid L2-II-1, and the structure D is DOTA.

7. The compound of formula D-L2(-B)-L1-A or a pharmaceutically acceptable salt thereof of claim 6, wherein the structure of the compound of formula D-L2(-B)-L1-A or a pharmaceutically acceptable salt thereof is shown below: 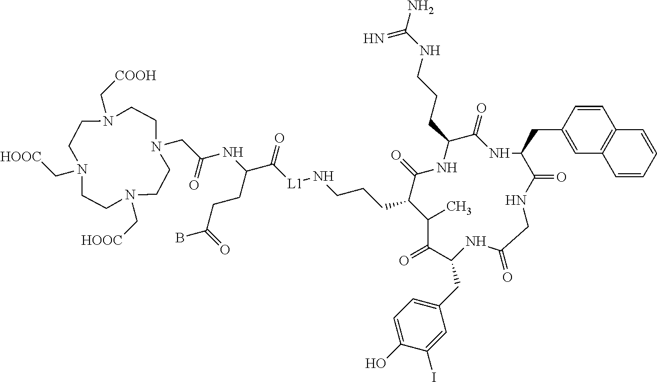
wherein the L1 structure is of type L1-I, and a carboxylic acid group is provided at one end, and an amino group is provided at the other end, and the carboxylic acid group end forms an amide bond with structure A, wherein L1-I is selected from one of the group consisting of molecular structures 1 to 26 listed in Table 1;
wherein B is the structure of type B-II having an amino group, wherein B—II is selected from one of the group consisting of molecular structures 1 to 11 listed in Table 6.

8. The compound of formula D-L2(-B)-L1-A or a pharmaceutically acceptable salt thereof of claim 3, wherein the B structure is 4-(p-Tolyl) butyric acid B-I-01.

9. The compound of formula D-L2(-B)-L1-A or a pharmaceutically acceptable salt thereof of claim 8, wherein the L1 structure is 4-(aminomethyl)benzoic acid L1-I-01 or 6-aminohexanoic acid L1-I-17.

10. The compound of formula D-L2(-B)-L1-A or a pharmaceutically acceptable salt thereof of claim 3, wherein the B structure is N-(2-aminoethyl)-4-methyl-benzenebutanamide B-II-01.

11. The compound of formula D-L2(-B)-L1-A or a pharmaceutically acceptable salt thereof of claim 10, wherein the L1 structure is 1,4-butane dicarboxylic acid L1-II-01 or 1,4-benzenedicarboxylic acid L1-II-08.

12. The compound of formula D-L2(-B)-L1-A or a pharmaceutically acceptable salt thereof of claim 3, wherein the compound of formula D-L2(-B)-L1-A or a pharmaceutically acceptable salt thereof comprises the following compound structures:

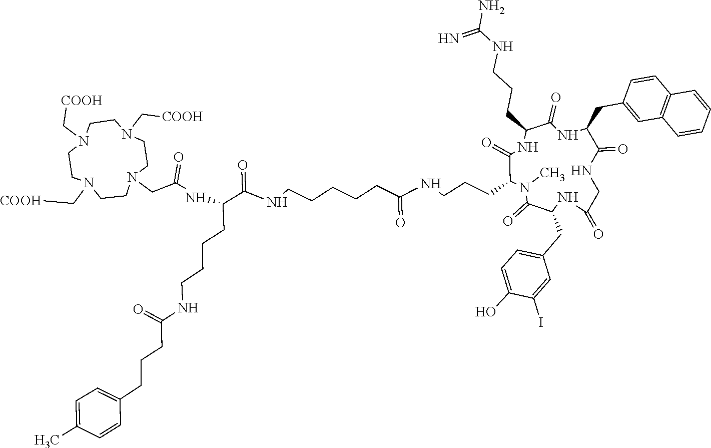 [SMILES: Cc1ccc(CCCC(=O)NCCCC[C@H](NC(=O)CN2CCN(CC(=O)O)CCN(CC(=O)O)CCN(CC(=O)O)CC2)C(=O)NCCCCCC(=O)NCCC[C@@H]2C(=O)N[C@@H](CCCNC(=N)N)C(=O)N[C@@H](Cc3ccc4ccccc4c3)C(=O)NCC(=O)N[C@H](Cc3ccc(O)c(I)c3)C(=O)N2C)cc1]

| Nomenclature | I-01(-I-01)-I-17 |
|---|---|
| Synthesis scheme | 1 |
| | | | |
|---|---|---|---|
| D | L2 | B | L1 |
| DOTA | L2-I-01 | B-I-01 | L1-I-17 |

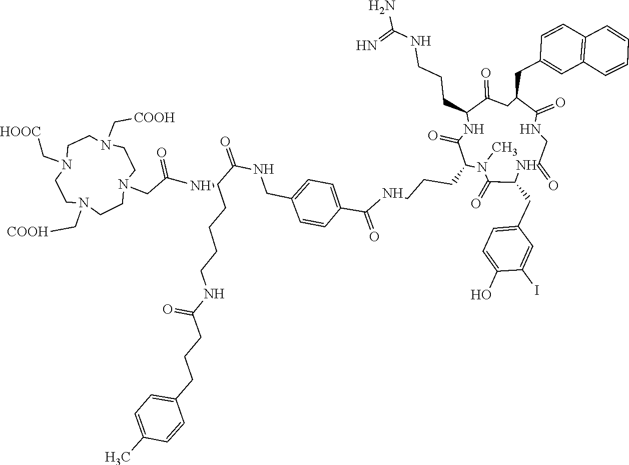 [SMILES: Cc1ccc(CCCC(=O)NCCCC[C@H](NC(=O)CN2CCN(CC(=O)O)CCN(CC(=O)O)CCN(CC(=O)O)CC2)C(=O)NCc2ccc(C(=O)NCCC[C@@H]3C(=O)N[C@@H](CCCNC(=N)N)C(=O)C[C@@H](Cc4ccc5ccccc5c4)C(=O)NCC(=O)N[C@H](Cc4ccc(O)c(I)c4)C(=O)N3C)cc2)cc1]
| Nomenclature | I-01(-I-01)-I-01 | | |
|---|---|---|---|
| Synthesis scheme | 1 | | |
| D | L2 | B | L1 |
| DOTA | L2-I-01 | B-I-01 | L1-I-01 |

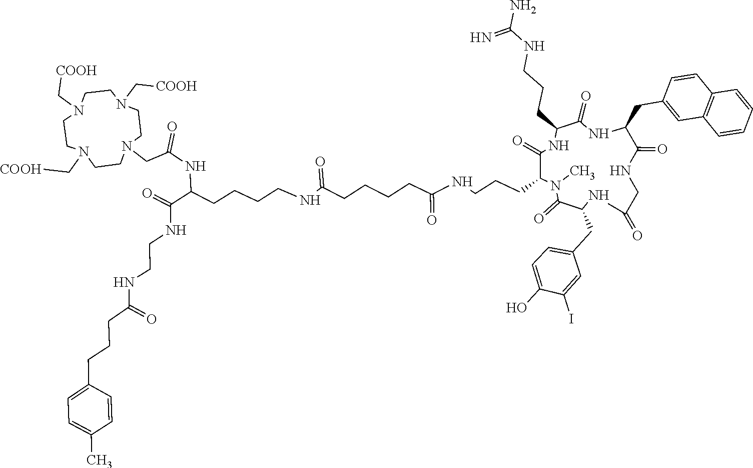 [SMILES: Cc1ccc(CCCC(=O)NCCNC(=O)C(CCCCNC(=O)CCCCC(=O)NCCC[C@@H]2C(=O)N[C@@H](CCCNC(=N)N)C(=O)N[C@@H](Cc3ccc4ccccc4c3)C(=O)NCC(=O)N[C@H](Cc3ccc(O)c(I)c3)C(=O)N2C)NC(=O)CN2CCN(CC(=O)O)CCN(CC(=O)O)CCN(CC(=O)O)CC2)cc1]
| Nomenclature | I-01(-II-01)-II-01 | | |
|---|---|---|---|
| Synthesis scheme | 2 | | |
| D | L2 | B | Ll |
| DOTA | L2-I-01 | B-II-01 | L1-II-01 |

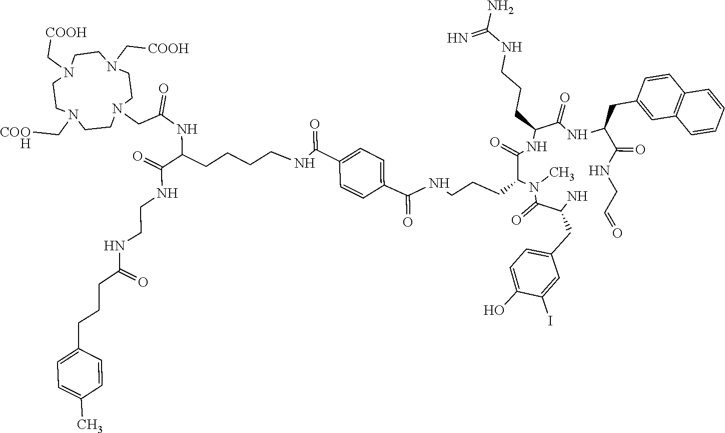 [SMILES: Cc1ccc(CCCC(=O)NCCNC(=O)C(CCCCNC(=O)c2ccc(C(=O)NCCC[C@H](C(=O)N[C@@H](CCCNC(=N)N)C(=O)N[C@@H](Cc3ccc4ccccc4c3)C(=O)NCC=O)N(C)C(=O)[C@H](N)Cc3ccc(O)c(I)c3)cc2)NC(=O)CN2CCN(CC(=O)O)CCN(CC(=O)O)CCN(CC(=O)O)CC2)cc1]
| Nomenclature | I-01(-II-01)-II-08 | | |
|---|---|---|---|
| Synthesis scheme | 2 | | |
| D | L2 | B | Ll |
| DOTA | L2-I-01 | B-II-01 | L1-II-08. |

13. The compound of formula D-L2(-B)-L1-A or a pharmaceutically acceptable salt thereof of claim 5, wherein the B structure is N-(-I-aminophenyl)-4-methyl-benzenebutanamide B-II-08.

14. The compound of formula D-L2(-B)-L1-A or a pharmaceutically acceptable salt thereof of claim 13, wherein the L1 structure is 6-aminohexanoic acid L1-I-17.

15. The compound of formula D-L2(-B)-L1-A or a pharmaceutically acceptable salt thereof of claim 5, wherein the compound of formula D-L2(-B)-L1-A or a pharmaceutically acceptable salt thereof comprises the following compound structures:

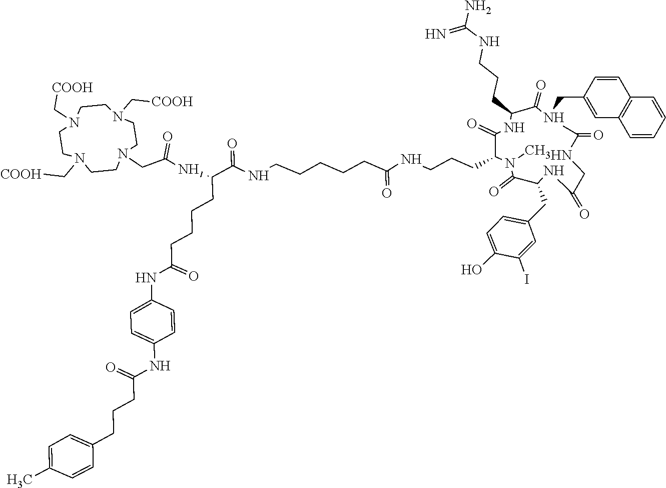

| Nomenclature | II-03(-11-08)-I-17 |
|---|---|
| Synthesis scheme | 3 |
| | | | |
|---|---|---|---|
| D | L2 | B | Ll |
| DOTA | L2-II-03 | B-II-08 | L1-I-17. |

16. The compound of formula D-L2(-B)-L1-A or a pharmaceutically acceptable salt thereof of claim 7, wherein the structure B is N-(2-aminoethyl)-4-methyl-benzenebutanamide B-II-01.

17. The compound of formula D-L2(-B)-L1-A or a pharmaceutically acceptable salt thereof of claim 16, wherein the L1 structure is 1,4-butane dicarboxylic acid L1-II-01.

18. The compound of formula D-L2(-B)-L1-A or a pharmaceutically acceptable salt thereof of claim 7, wherein the compound of formula D-L2(-B)-L1-A or a pharmaceutically acceptable salt thereof comprises the following compound structures:

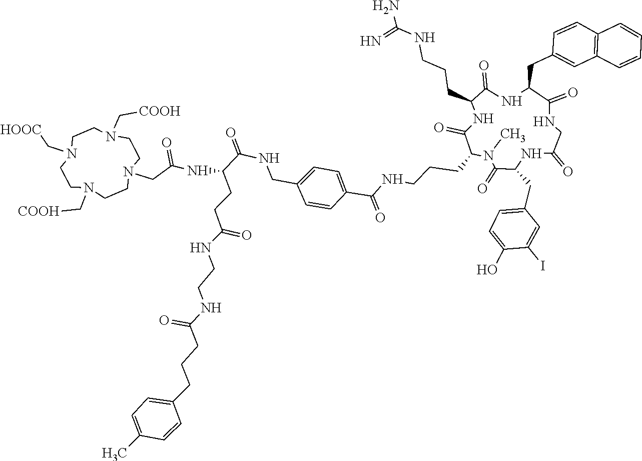 [SMILES: Cc1ccc(CCCC(=O)NCCNC(=O)CC[C@H](NC(=O)CN2CCN(CC(=O)O)CCN(CC(=O)O)CCN(CC(=O)O)CC2)C(=O)NCc2ccc(C(=O)NCCC[C@@H]3C(=O)N[C@@H](CCCNC(=N)N)C(=O)N[C@@H](Cc4ccc5ccccc5c4)C(=O)NCC(=O)N[C@H](Cc4ccc(O)c(I)c4)C(=O)N3C)cc2)cc1]

| Nomenclature | II-01(-II-01)-1-01 |
|---|---|
| Synthesis scheme | 3 |
| | | | |
|---|---|---|---|
| D | L2 | B | L1 |
| DOTA | L2-II-01 | B-II-01 | L1-I-01. |

19. The compound of formula D-L2(-B)-L1-A or a pharmaceutically acceptable salt thereof of claim 1, wherein the chelator structure D capable of binding with metal ions comprise any one ofs type D-1 to D-6:

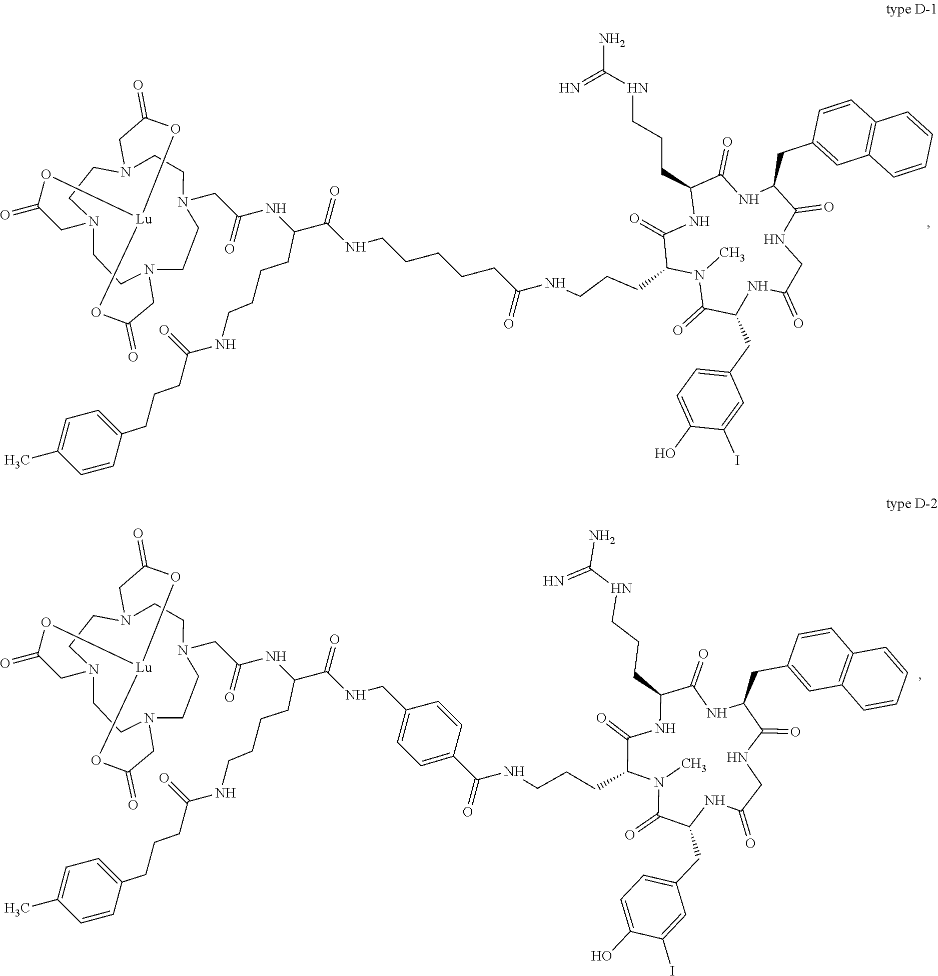

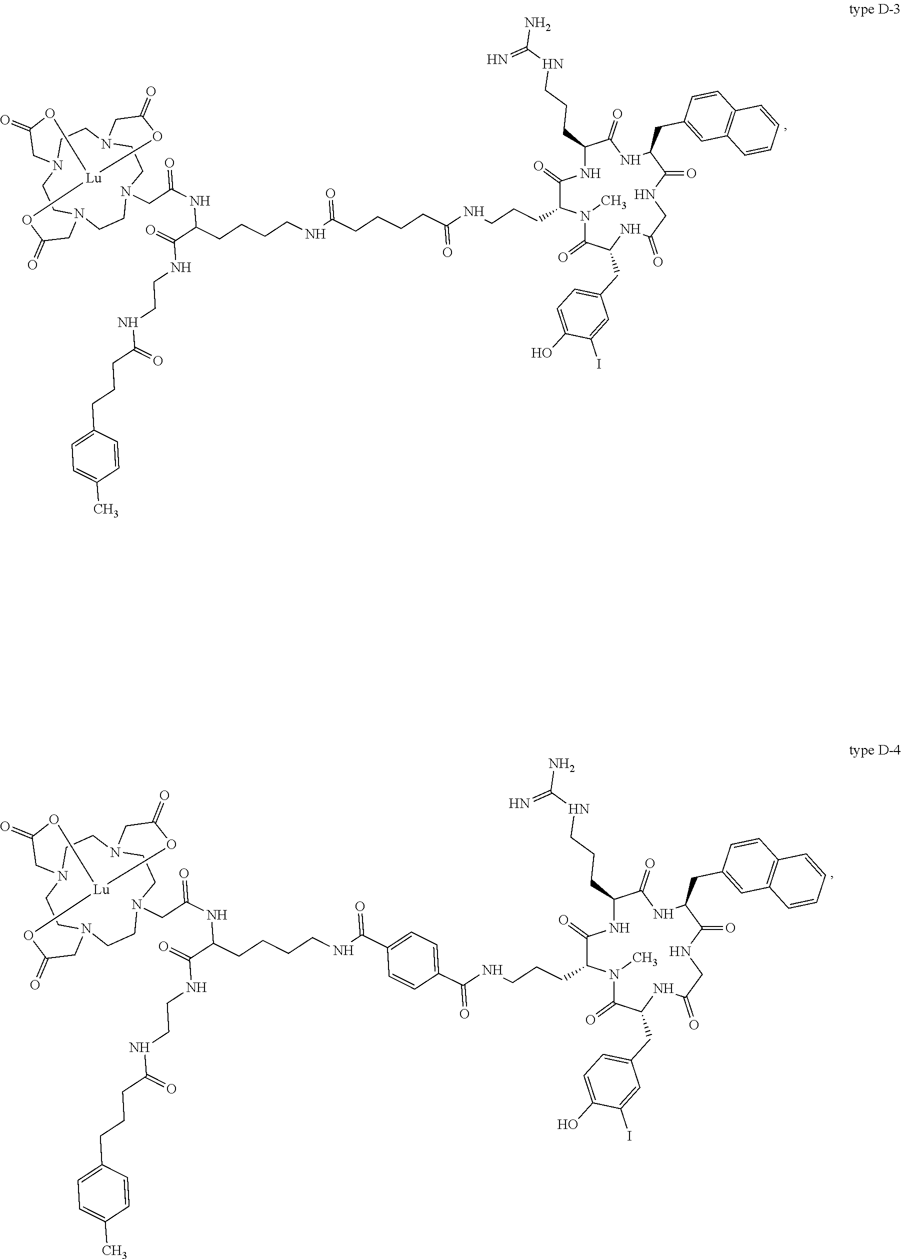

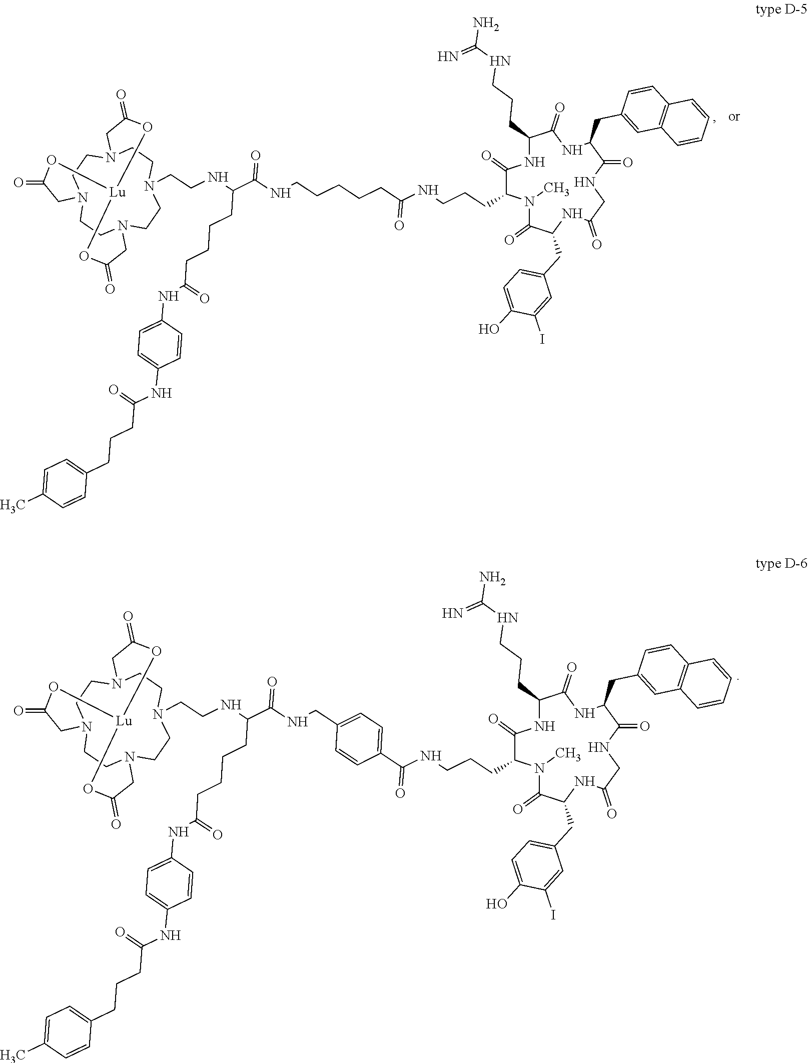

20. A method for producing the compound of formula D-L2(-B)-L1-A or a pharmaceutically acceptable salt thereof of claim 1, wherein the synthesis schemes comprises: 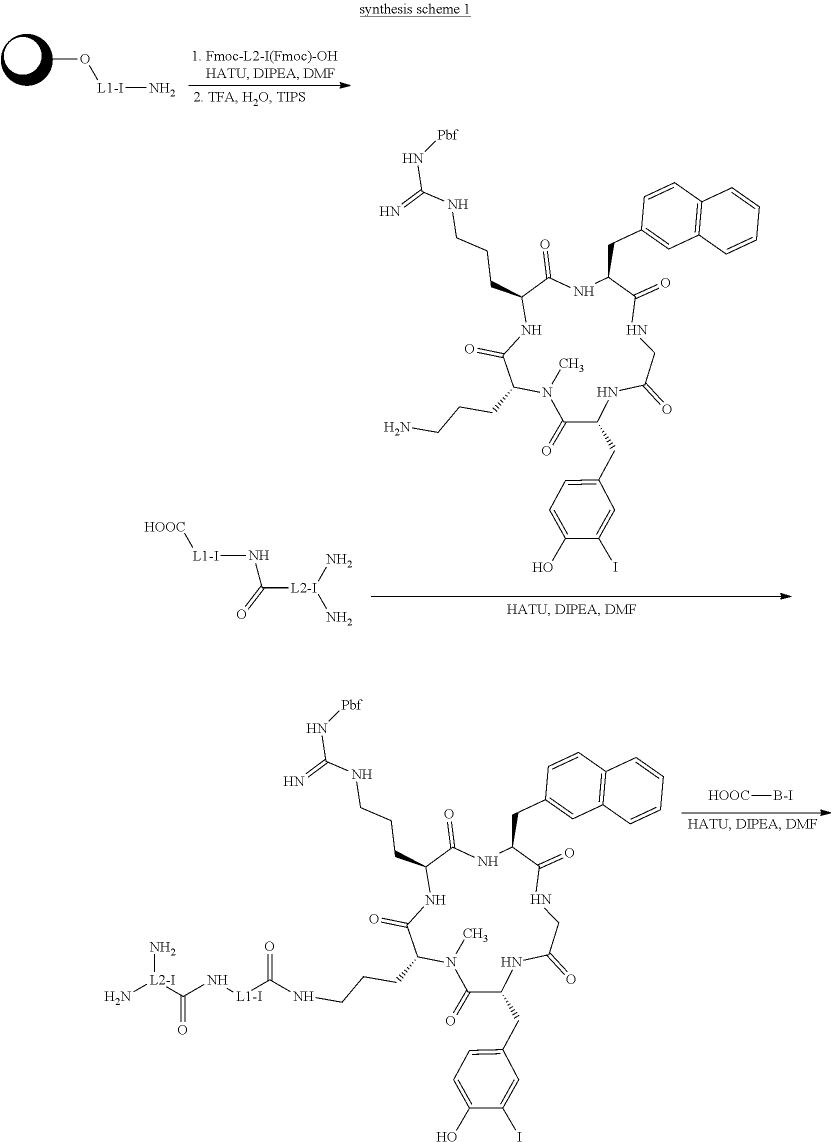 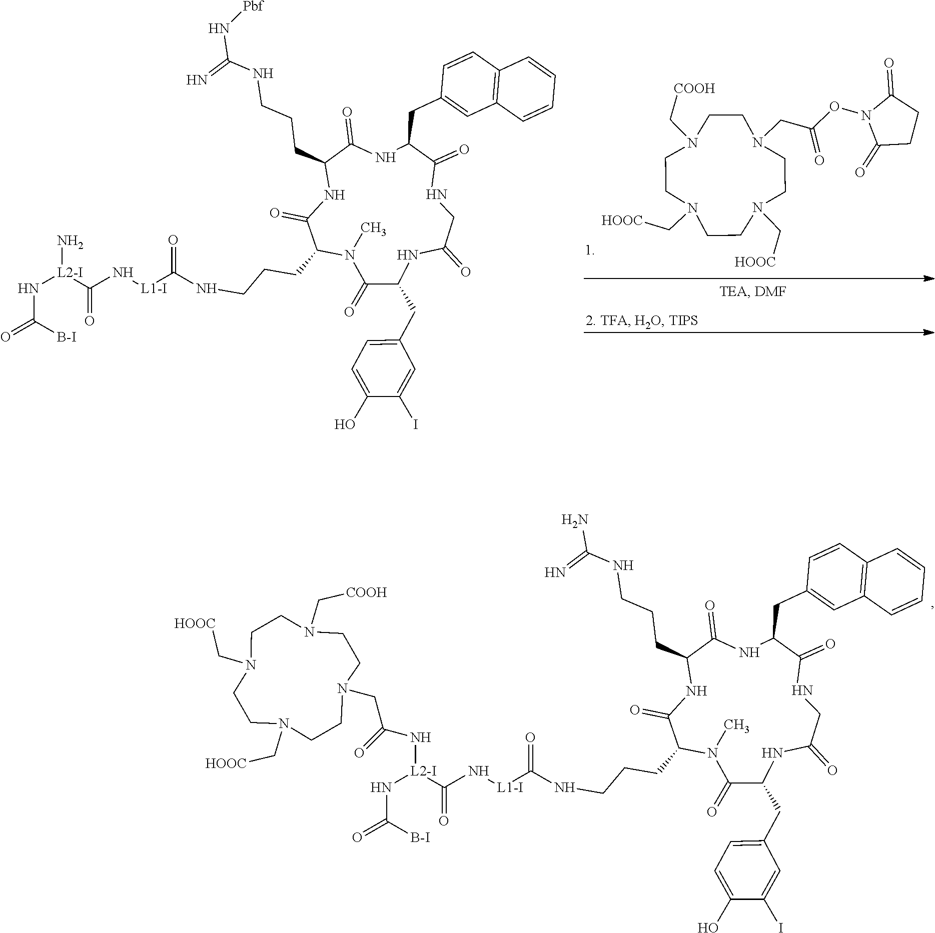 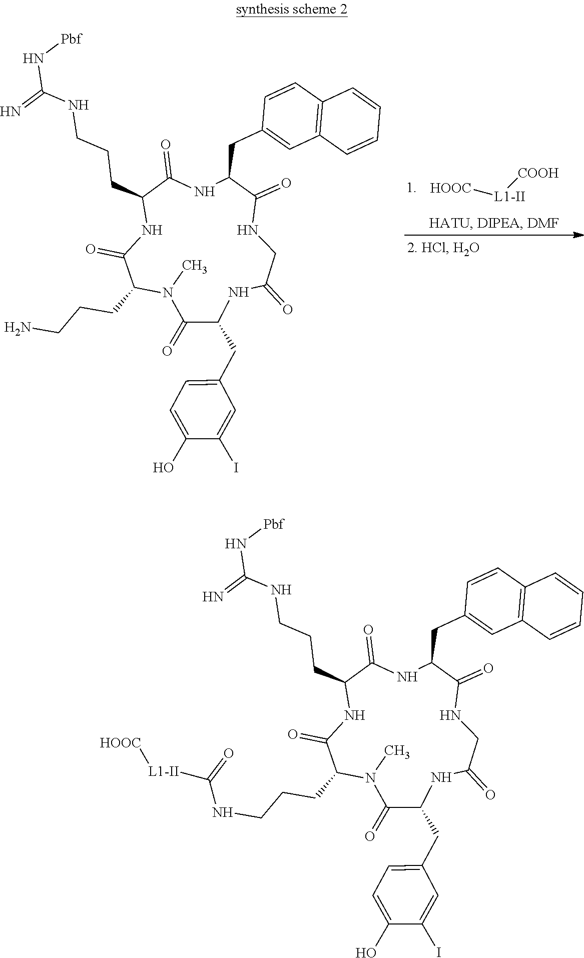 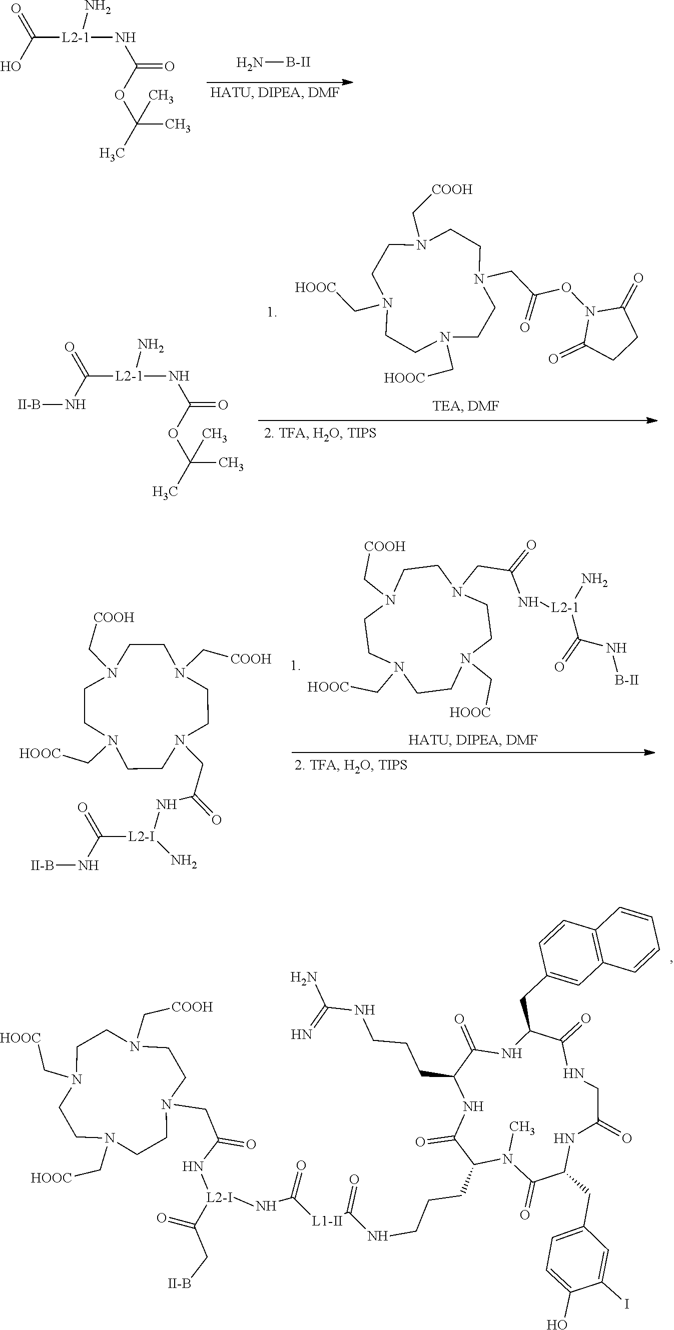 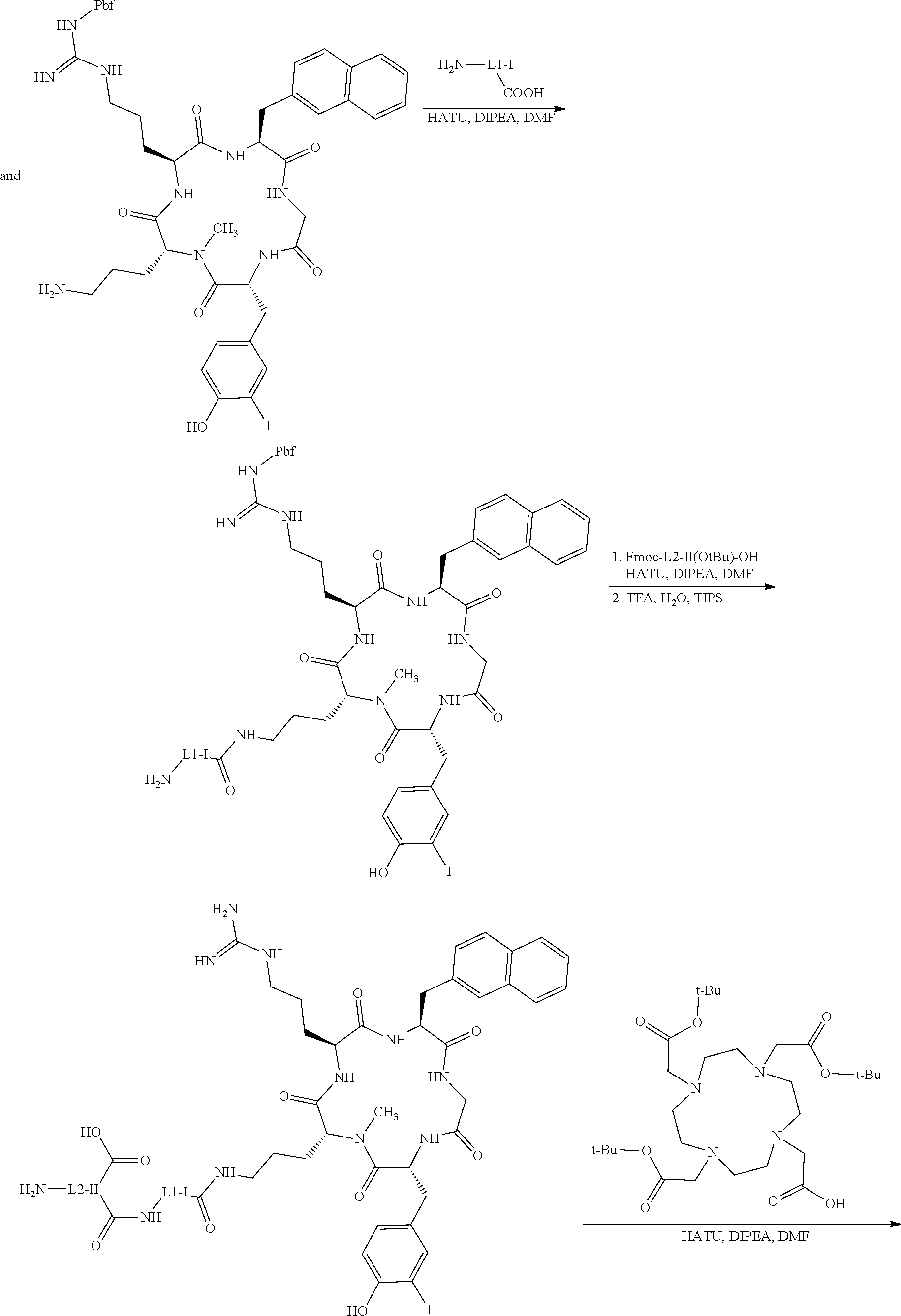 [chemical structure]
